(19) United States
(12) Patent Application Publication
(10) Pub. No.: US 2024/0018576 A1
(43) Pub. Date: Jan. 18, 2024

(54) SPATIAL RNA LOCALIZATION METHOD

(71) Applicant: REBUS BIOSYSTEMS, INC., Santa Clara, CA (US)

(72) Inventors: Sten LINNARSSON, Santa Clara, CA (US); Simone CODELUPPI, Santa Clara, CA (US); Lars BORM, Santa Clara, CA (US)

(21) Appl. No.: 18/356,134

(22) Filed: Jul. 20, 2023

Related U.S. Application Data
(63) Continuation of application No. PCT/US2022/013192, filed on Jan. 20, 2022.
(60) Provisional application No. 63/139,701, filed on Jan. 20, 2021.

(51) Int. Cl. C12Q 1/6841; C12Q 1/6837

(52) U.S. Cl. CPC C12Q 1/6841; C12Q 1/6837; C12Q 1/6806

(57) ABSTRACT

Provided herein is a method for imaging RNA transferred from a sample to a substrate. The method may include placing a sample comprising cells on a substrate, transferring RNA from the sample onto the substrate to produce an RNA blot in which the RNA is immobilized on the substrate, removing the sample from the substrate, hybridizing the RNA blot with a set of oligonucleotides that hybridize to different sites in the same RNA species, and reading the blot to obtain an image showing the binding pattern of the hybridized oligonucleotides.

### RELATED APPLICATIONS

This application is a continuation application of International Patent Application Serial No. PCT/US2022/013192, filed Jan. 20, 2022, which claims the benefit of, and priority to, U.S. Provisional Patent Application Ser. No. 63/139,701, filed Jan. 20, 2021, both of which are incorporated by reference herein in their entireties.

### BACKGROUND

In conventional single molecule fluorescence in situ hybridization (smFISH) methods, individual ribonucleic acid (RNA) molecules are detected by hybridizing a tissue section with multiple short deoxyribonucleic acid (DNA) probes that are complementary to the target RNA and conjugated to a fluorescent dye. Binding of a single probe results in weak signal, but the signal from the ensemble of all of the probes is robust. Because of its ability to detect single RNA molecules, smFISH has become a powerful technique for studying gene expression in single cells. For example, smFISH allows measurement of the cell-to-cell variability in gene expression and determination of intracellular RNA localization. Further, multiplexed versions of smFISH can be used to analyze the expression of tens or hundreds of genes (see Codeluppi et al., Nature Methods 2018 15: 932-935).

The problem with conventional smFISH methods (and their multiplexed brethren such as the osmFISH, as described by Codeluppi 2018) is that their throughput is severely limited by the diffraction limit of light and the numerical aperture of the objective lenses that are typically used in lab microscopes. Specifically, at the magnification required to resolve individual RNA molecules, the field of view and depth of field of the microscope become so narrow and shallow that the full depth of the entire sample cannot be imaged at once. This problem has been solved by taking thousands or even tens of thousands of stacks of images across each tissue section, where each image in a stack is taken at a different depth in the sample (i.e., in the “z” dimension). The stacked images are then “flattened”, i.e., combined into one in the z dimension, and then joined together with other flattened images, side-by-side. Illustrated by example, if an objective lens has a depth of field of −0.300 nm, a single area (a single field of view) in a 10-um tissue section will need to be imaged over 30 times, each time at a different depth, to produce over 30 pancaked images of the area (referred to as a “Z-stack”). Each Z-stack is then flattened to produce a tile and then stitched together with thousands or tens of thousands of other tiles to produce a mosaic. Since this process (z-stacking and flattening) needs to occur for each tile and for each gene, some smFISH experiments are enormously time consuming. For example, Codeluppi (2018) described a multiplexed smFISH method in which the expression patterns of 33 genes were analyzed. Codeluppi's experiment took two weeks to perform. Analysis of 150 or more genes by smFISH using the same method could therefore take several months.

It follows from the above that smFISH and multiplexed smFISH require specialized hardware (e.g., a microscope that is capable of stepping in the z-plane, to take z-stacks) and software (for flattening the Z-stacks) and are limited by the enormous amount of time that it takes to complete a single experiment.

The methods and devices described herein can solve at least these problems.

### SUMMARY

Provided herein is a method for imaging RNA transferred from a sample to a planar substrate. In some embodiments, the method includes placing a sample comprising cells and having at least one planar surface on a planar substrate, transferring RNA from the sample onto the planar substrate to produce an RNA blot in which the RNA is immobilized on the substrate, removing the sample from the substrate, hybridizing the RNA blot with a set of oligonucleotides that hybridize to different sites in the same RNA species, and reading the blot to obtain an image showing the binding pattern of the hybridized oligonucleotides.

In any embodiment, the RNA may be transferred to the planar substrate by electrophoresis, which increases the transfer efficiency and reduces the distortion that can be sometimes introduced by other methods. In these embodiments, the RNA may be transferred from the sample to the planar sample by: i. placing the sample on a planar, optically transparent, conductive substrate, ii. positioning a planar electrode opposite to the sample and iii. applying a voltage across the substrate and electrode when the sample immersed in a conductive liquid, thereby moving the RNA in the sample to the substrate.

In the present method the RNA is transferred to a two-dimensional surface prior to microscopy. The transfer step provides a variety of advantages, some of which are described below.

Depending on how the method is performed, the transfer step virtually eliminates the need for a full Z-stack through the tissue which, in turn, tremendously increases the throughput of the method. Further, because the RNA has been transferred to a planar surface, the depth of field of the microscope used to image the RNA can be very shallow. This, in turn, increases the magnification and the resolution of the method. In some implementations, the resolution of the present method is easily in the range of 130-270 nm for probes that are labeled with a moiety that emits a signal in the visual spectrum of light. Thus, some embodiments of the present method can be practiced without a microscope that can step through the z-dimension. A standard fluorescent microscope can be used in some cases.

In addition, removal of the tissue prior to microscopy removes a source of background, which, in turn provides data with higher signal to noise ratios than conventional methods. A higher signal-to-noise ratio increases the sensitivity of the assay and allows the exposure times to be shorter, thereby speeding up the imaging steps of the method.

In addition, because the target RNA (and/or any primary oligonucleotide probes hybridized thereto) are affixed on a support and there is no tissue for the probes or other reagents to diffuse through a tissue (which can be quite dense), the kinetics of the downstream reactions (e.g., probe hybridization, probe inactivation/removal and washing steps) are greatly improved. In other words, the RNA and/or any primary oligonucleotide probes hybridized to the RNA are more accessible to the reagents that are in solution (e.g., the probes and other chemicals) since those reagents can reach those molecules directly, without having to diffuse through a dense tissue to reach the RNA. Any wash steps (in which unnecessary reagents or reactants are removed) should be more efficient for the same reason.

In some embodiments (e.g., those in which electrophoresis is used to transfer the RNA from the tissue to the slide), electrophoresis can be readily used to increase the local concentration of probes (e.g., primary oligonucleotide probes or labeled probes) at the surface of the slide, where they can hybridize to their targets much more efficiently and quickly.

In accordance with some embodiments, a method includes receiving a substrate with a layer of one or more cells thereon; and transferring nucleic acids within the one or more cells toward the surface.

In accordance with some embodiments, an apparatus includes a mount for receiving a substrate; and an electrical source positioned adjacently to the mount for providing one or more electrical fields in a direction that is substantially perpendicular to the substrate.

### BRIEF DESCRIPTION OF THE FIGURES

The skilled artisan will understand that the drawings, described below, are for illustration purposes only. The drawings are not intended to limit the scope of the present teachings in any way.

FIG. 1A: Schematic illustration of some principles of the method.

FIG. 1B: Schematic illustration of a device for inducing an electrical force in accordance with some embodiments.

FIG. 1C: Schematic illustration of another arrangement of electrodes.

FIG. 2: Schematic illustration of a labeling protocol that can be used in the present method.

FIG. 3: Illustration of the electrophoresis setup used in the present method.

FIG. 4: Images of mouse brain. Top panel: expression of 167 genes. Bottom panel:

brain atlas generated by the Allen Institute.

FIG. 5: Images of individual genes obtained from the present method (left) compared to those published in the brain atlas.

FIG. 6: Signals from individual genes shown at two different magnifications. The clustering of the signals indicates that the data are at a single cell resolution.

FIG. 7: Overlay of data with nuclei image (shown in white). These data confirm that the signals are at a single cell resolution.

FIG. 8: Image of a that is similar to that shown in FIG. 5, but the RNA was transferred by diffusion alone, i.e., without electrophoresis.

FIG. 9: Side-by-side images of cerebellum, where the RNA was transferred by electrophoresis on the left and the RNA was transferred by diffusion alone on the right.

FIG. 10: Close-up side-by-side images of cerebellum, where the RNA was transferred by electrophoresis on the left and the RNA was transferred by diffusion alone on the right.

FIG. 11: shows a comparison of results that are obtained using “regular” primary oligonucleotide probes (e.g., probes that do not have a 5′ amine group) with primary oligonucleotide probes that have a 5′ amine.

FIG. 12: shows total gene count comparison of results that are obtained using “regular” primary oligonucleotide probes, i.e., probes that do not have a 5′ amine group with primary oligonucleotide probes that have a 5′ amine.

FIG. 13: shows probability distributions for spots where the barcode is present and for spots where the barcode is absent.

FIG. 14: shows a flow diagram illustrating a method of transferring nucleic acids in accordance with some embodiments.

### DEFINITIONS

Unless defined otherwise herein, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

All patents and publications, including all sequences disclosed within such patents and publications, referred to herein are expressly incorporated by reference.

Numeric ranges are inclusive of the numbers defining the range. Unless otherwise indicated, nucleic acids are written left to right in 5′ to 3′ orientation; amino acid sequences are written left to right in amino to carboxy orientation, respectively.

The headings provided herein are not limitations of the various aspects or embodiments of the invention. Accordingly, the terms defined immediately below are more fully defined by reference to the specification as a whole.

Unless defined otherwise, all technical and scientific terms used herein have the same meaning as commonly understood by one of ordinary skill in the art to which this invention belongs. Singleton, et al., DICTIONARY OF MICROBIOLOGY AND MOLECULAR BIOLOGY, 2D ED., John Wiley and Sons, New York (1994), and Hale & Markham, THE HARPER COLLINS DICTIONARY OF BIOLOGY, Harper Perennial, N.Y. (1991) provide one of skill with the general meaning of many of the terms used herein. Still, certain terms are defined below for the sake of clarity and ease of reference.

As used herein, the term “sample comprising cells and having at least one planar surface” refers to a sample that has at least one side that has a substantially planar, i.e., two-dimensional surface, where the sample contains cells. Such a sample can be made by, e.g., growing cells on a planar surface, depositing cells on a planar surface, e.g., by centrifugation, by cutting a three-dimensional object that contains cells into sections. The sample may be fresh, fresh frozen and it may be unfixed or fixed. If the sample is fixed, it may be fixed using any number of reagents including formalin, methanol, paraformaldehyde, methanol:acetic acid, glutaraldehyde, bifunctional crosslinkers such as bis(succinimidyl)suberate, bis(succinimidyl)polyethylene glycol etc. A section (e.g., a cryosection) of a tissue sample (e.g., of a fresh frozen tissue sample) that has a thickness in the range of 1-50 um (e.g., in the range of 1-5 um or 5-20 um) is an example of a sample comprising cells and having at least one planar surface, although there are many alternatives.

As used herein, the term “planar substrate” refers to a substrate having a substantially flat surface and that is compatible with microscopy. Microscope slides (which are made of glass) and coated microscope slides are examples of such substrates.

As used herein, the term “RNA blot” or “blot” refers to product in which the RNA molecules from a sample are spatially arranged on a substate in a way that correlates with the arrangement of those molecules in the sample. As noted below, in transferring RNA from a sample to a substrate, some lateral diffusion may occur and, as such, an RNA blot may reflect any lateral diffusion of the molecules. Thus, the pattern of RNA molecules on the blot may be at a lower resolution than they actually are in the sample. However, as will be described below, in many embodiments the RNA may be transferred from the sample to the substrate at a single-cell resolution, meaning that on the blot the RNA molecules from a particular cell should be spatially separate from the RNA molecules from a neighboring cell (in the x-y plane).

As used herein, the term “immobilized” means that the RNA molecules may be tethered to the surface of the substrate covalently or non-covalently, or any combination thereof. For example, the RNA molecules can be immobilized via electrostatic interactions (e.g., with polylysine, polyglutamic acid or a copolymer of the same), base-pairing (e.g., to oligo(dT), which is pre-fixed to the substrate), adsorption (see Lui et al Langmuir 2015 31 1: 371-377) or a covalent reaction between a group on the surface of the substrate (e.g., an epoxy ring) and the RNA. In addition to any of these interactions, the RNA may be cross-linked to the substrate after the tissue has been removed (e.g., using paraformaldehyde).

As used herein, the term “removing” refers to any action that results of the elimination of an object. Removing may include any combination of dissolving, degrading, destroying, washing away and/or peeling off (e.g., using tweezers).

As used herein, the term “oligonucleotide” refers to a multimer of at least 10, e.g., at least 15 or at least 30 nucleotides. In some embodiments, an oligonucleotide may be in the range of 15-200 nucleotides in length, or more. Any oligonucleotide used herein may be composed of G, A, T and C, or bases that are capable of base pairing reliably with a complementary nucleotide. The oligonucleotides used herein may contain natural or non-natural nucleotides or linkages and, in some embodiments, may be labeled. The oligonucleotides used in the present method are typically DNA (not RNA) oligonucleotides. Oligonucleotides can contain nucleotide analogs in any embodiment.

As used herein, the term “tail”, in the context of a tailed oligonucleotide, refers to a part of an oligonucleotide that is not complementary to a cellular RNA and does not hybridize to the cellular RNA. A tail can be at the 5′ end or 3′ end of an oligonucleotide. In some cases, an oligonucleotide may have a tail at both ends. A tail can be as long as needed, e.g., in the range of 20-100 bases, as desired.

As used herein, the term “reading” in the context of reading a fluorescent signal, refers to obtaining an image by scanning or by microscopy, where the image shows the pattern of fluorescence as well as the intensity of fluorescence.

As used herein, the term “multiplexing” refers to an analysis of RNAs encoded by more than one gene.

A “plurality” contains at least 2 members. In certain cases, a plurality may have at least 2, at least 5, at least 10, at least 100, at least 1000, at least 10,000, at least 100,000, at least 10⁶, at least 10⁷, at least 10⁸ or at least 10⁹ or more members. As used in the description of the various described embodiments and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof. The term “exemplary” is used herein in the sense of “serving as an example, instance, or illustration” and not in the sense of “representing the best of its kind.”

Other definitions of terms may appear throughout the specification.

### DETAILED DESCRIPTION

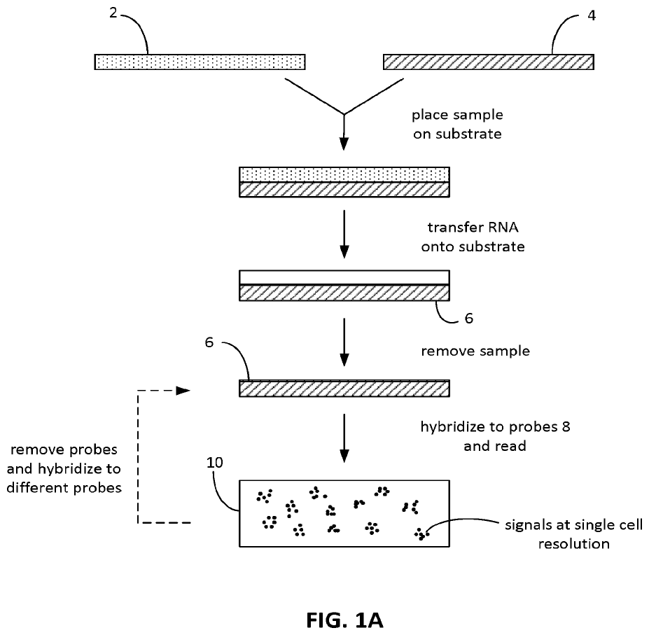

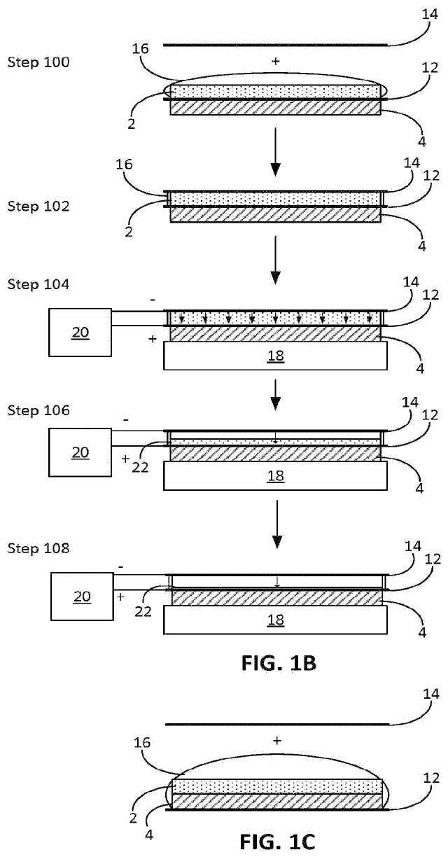

Provided herein is a method analyzing a sample. Some principles of the method are shown in FIG. 1A. With reference to FIG. 1A, some embodiments of the method include comprise placing a sample 2 (e.g., a tissue section) comprising cells and having at least one planar surface on a planar substrate 4 (with the planar surfaces facing each other), transferring RNA from the sample onto the planar substrate to produce an RNA blot 6 in which the transferred RNA is immobilized on the substrate, removing the sample from the substrate, hybridizing the RNA blot with a set of oligonucleotides 8 that hybridize to different sites in the same RNA species, and reading the blot to obtain an image 10 showing the binding pattern of the hybridized oligonucleotides. As would be apparent, the cells in the sample can be permeabilized after the sample has been placed on the substrate, but before the RNA is transferred. Permeabilization can be done using a variety of permeabilization agents such as a detergent (e.g., triton-X100 or Tween 20) or a solvent such as ethanol or acetone. The permeabilization may be done in the presence of a reducing agent (e.g., DTT).

In some embodiments, the RNA is transferred to the planar substrate by electrophoresis. However, as shown in the experimental section below, the RNA can also be transferred to the substrate by other methods, e.g., diffusion and/or electrostatic attraction. In embodiments that employ electrophoresis (e.g., as shown in FIG. 1B), the RNA may be transferred from the sample to the planar substrate by sandwiching the sample, in a conductive liquid (e.g., conductive liquid 16), between two planar electrodes (e.g., electrodes 12 and 14) and applying a voltage (e.g., using an electrical source 20, such as a voltage source like a battery or a AC-to-DC converter) that moves the RNA away from one electrode towards the other (e.g., RNA 22 in the sample 2 moves away from the electrode 14 and toward the electrode 12). In some embodiments, the planar substrate may be used as both an electrode and a microscope slide (e.g., as shown in FIG. 1B). In these embodiments, the transfer may be done by: i. placing the sample on a planar, optically transparent (i.e., transparent in the wavelengths of light being used during microscopy), conductive substrate (e.g., step 100), ii. positioning a planar electrode opposite to the sample (e.g., step 102), and iii. applying a voltage across the substrate and electrode (e.g., step 104) when the sample immersed in a conductive liquid, e.g., water or a buffer such as TAE, TBE or sodium borate buffer, thereby moving the RNA in the sample to the substrate (which is the positive electrode for this process in some embodiments), as shown steps 106 and 108. In these embodiments, the electrophoresis medium (e.g., the conductive liquid) optionally comprises a chemical denaturant (e.g., urea, formamide, DMSO or a chaotropic agent) in order to denature the RNA, inactivate proteins that may degrade the RNA, and remove any RNA binding proteins. In some embodiments, the size of the gap between the electrodes and voltage differential between the electrodes is not critical. In some embodiments, the gap between the electrodes should be greater than the thickness of the sample (e.g., in the range of 0.5 mm to 2 mm). A voltage of 15 V/cm gap has been successfully used, and it is expected that any voltage in the range of 5 V/cm to 100 V/cm or other voltages outside of this range could also be used. In these embodiments, the substrate itself may be examined by microscopy, without having to remove or separate any parts from the substrate.

In some embodiments, the electrode 12 is located between the electrode 14 and the substrate 4, as shown in FIG. 1B. In some embodiments, the substrate 4 is located between the electrode 12 and the electrode 14, as shown in FIG. 1C.

In electrophoresis embodiments, the substrate may be a transparent (e.g., glass) slide coated in a transparent conductive metal oxide (TCO), or a thin layer of gold, titanium with gold, chromium with gold. For example, the TCO coating may be an indium tin-oxide (ITO), aluminum-doped zinc oxide (AZO), indium-doped cadmium oxide ICO), molybdenum indium oxide, (MIO), gallium zinc oxide (GZO), fluorine-doped indium oxide (IFO) or indium zinc oxide (IZO) coating, methods for the fabrication of which are known (see, e.g., Chen et al Langmuir 2013 29: 13836-13842). In some embodiments, the surface of the substrate may be modified prior to placement of the sample on it. For example, the surface may be treated with a bifunctional organosilane (e.g., (3-Glycidyloxypropyl) trimethoxysilane; GPTMS) and then optionally coated with oligo(dT) or polycationic adhesive such as polylysine. The sample may be placed directly or indirectly on the substrate. For clarity, the substrate does not contain an array or lawn of oligonucleotides (except in the case where oligo(dT) may be used).

The RNA may become immobilized on the surface of the substrate via any of a number of different chemistries, or a combination of the same. For example, RNA may become immobilized via base-pairing with oligo(dT), reacting with a functional group that has been added to the surface (e.g., using a bifunctional organosilane such as 3-aminopropyl triethoxysilane (APTES) or 3-glycidyloxypropyl trimethoxysilane (GPTMS)), by direct absorption to the conductive metal oxide (e.g., to ITO; see Lui et al Langmuir 2015 31 1: 371-377), or via an interaction with a polycationic adhesive such as polylysine. Alternative methods in which a reactive group is added to the RNA prior to immobilization may also be employed. For example, in some embodiments, the substrate may be a glass slide that has a surface of ITO that has been coated in oligo(dT) and poly-D-lysine.

After the RNA has been transferred to the substrate to produce a blot (e.g., a profile of RNA molecules positioned over a surface of the substrate), the sample is removed. In some embodiments, the sample may be digested with a protease (e.g., proteinase K) and washed away in a buffer that contains a denaturing detergent such as sodium dodecyl sulfate (SDS). However, the sample may also be removed by peeling it from the substate (without digestion) using tweezers or by washing away with a vigorous wash.

After removal of the sample, the RNA may be optionally crosslinked to the substrate (e.g., using PFA, for example), and washed. After washing, the RNA blot can be probed.

In some embodiments, the RNA blot is probed with labeled probes that hybridize directly to particular RNA species (e.g., an RNA encoded by a particular gene). In these embodiments, one or more sets of labeled probes that target a corresponding number of species of RNA are hybridized with the RNA blot under conditions by which the probes hybridize to the RNA on the blot. For example, each RNA species can be targeted by at least 10, 20, 30, 40 or 50 different, non-overlapping probes. Overlapping probes can also be used. After hybridization, the RNA blot may be imaged and, if desired, the hybridized probes can be removed or inactivated, and the RNA blot may be hybridized with one or more different sets of labeled probes (for different genes). These cycles can be repeated as necessary until sufficient data has been gathered. Such a probe system is described in Codeluppi et al (Nature Methods 2018 15: 932-935) and other methods that describe smFISH.

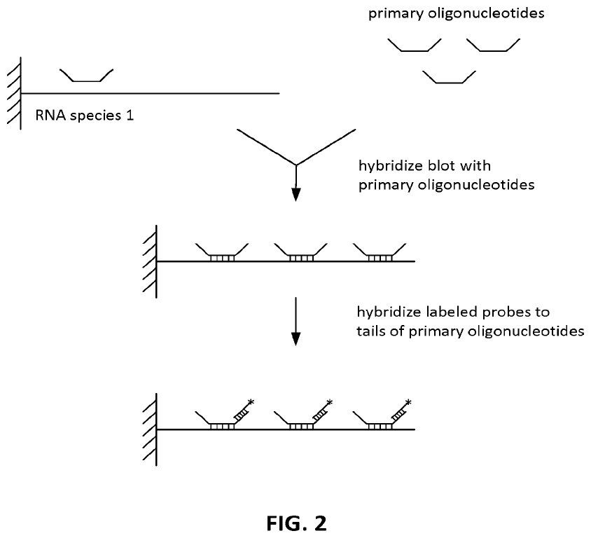

Alternatively, the hybridization and imaging steps may be implemented by hybridizing the RNA blot with one or more sets of unlabeled primary oligonucleotides that hybridize to different sites in the same RNA species and have a tail, hybridizing the blot with labeled probe that hybridizes to the tail of the unlabeled primary oligonucleotides and reading the blot to obtain an image showing the binding pattern of the hybridized labeled probes. This embodiment is shown in FIG. 2. Again, if more than one species is targeted then the tails of the primary oligonucleotides may be different and, in some cases the labeled probes that hybridize to those tails may be distinguishably labeled. The advantage of this implementation of the method is that fluorescent oligonucleotides can be quite expensive, and this implementation decreases the number of fluorescent oligonucleotides that are used in the method. In some embodiments, the method may be multiplexed by hybridizing the RNA blot with a set of unlabeled primary oligonucleotides en masse, and the RNAs to which the unlabeled primary oligonucleotides are bound are identified by multiple labeling, imaging and label inactivation cycles using a different subset of labeled probes each cycle. In these embodiments, the method includes: (a) obtaining multiple sets of unlabeled primary oligonucleotides and multiple labeled probes, wherein: i. each primary oligonucleotide comprises a sequence that hybridizes to a particular RNA species and a tail sequence that does not hybridize to the RNA; ii. the different sets of primary oligonucleotides hybridize to different RNA species; iii. the labeled probes hybridize to the tails of the primary oligonucleotides; iv. at least some of the labeled probes hybridize to multiple sets of primary oligonucleotides; and v. each set of primary oligonucleotides hybridizes with a unique combination of labeled probes; (b) placing a sample comprising cells and having at least one planar surface on a planar substrate; (c) transferring RNA from the sample onto the planar substrate to produce an RNA blot, wherein in the blot the RNA is immobilized on the substrate; (d) hybridizing the RNA blot, en masse, with the unlabeled primary oligonucleotides; (e) hybridizing the blot with a subset (e.g., 1, 2, 3 or 4) of the labeled probes; (f) reading the blot to obtain an image showing the binding pattern of the labeled probes hybridized in (e); (g) inactivating or removing the subset of labeled probes hybridized in (e), without removing the primary oligonucleotides; (h) repeating steps (e)-(f) using a different subset (e.g., 1, 2, 3 or 4) of the labeled probes (at least twice, at least 3 times, or at least 4 times e.g., 5-50 times), each repeat followed by step (g) except for the final repeat, to produce a plurality of images of the sample, each image corresponding to a subset of labeled probes hybridized in (e); (i) analyzing the same site in the plurality of images to identify which labeled oligonucleotides hybridized to the site; and (j) identifying the species of RNA at the site using the labeled oligonucleotides identified in (i). This last step may include use of a lookup table which contains a hybridization code (i.e., a list of probes that bind to the primary oligonucleotides corresponding to a single gene, or a code indicating the same) in one column and the name of a gene or code indicting the same in another column.

In other words, after removal or inactivation of a subset of probes in step (g), the blot may be hybridized with a different subset of the labeled probes (e.g., a second subset of the labeled probes, where the probes may be distinguishably labeled), and the sample may be re-read to produce an image showing the binding pattern for each of the most recently hybridized subset of probes. After that sample has been read, the probes may be removed from the sample and the hybridization and reading steps may be repeated with a different subset of distinguishably labeled probes. For example, the method may comprise repeating the hybridization, label removal/inactivation and reading steps multiple times with different subsets of labeled probes, each repeat is followed by removal of the probes (except for the final repeat), to produce a plurality of images of the sample, where each image corresponds to a subset of labeled nucleic acid probes. The hybridization/reading/label removal or inactivation steps can be repeated until all of the probes have been analyzed. Which spots correspond to which genes can be decoded by analysis of which labels bound to a particular spot.

In this description, the term “subset” is meant at least one, e.g., one, two, three or four, of a group or set of members, and the term “distinguishably labeled” means that the labels can be separately detected, even if they are at the same location. As such, in some embodiments, the method involves specifically hybridizing two, three or four of the labeled nucleic acid probes with the sample, thereby producing distinguishably labeled probe/oligonucleotide duplexes. As may be apparent, the sequence of the primary oligonucleotides that hybridize to the RNAs in in step (b) may be longer than the sequence of the probe that hybridizes to the primary oligonucleotides in step (e), thereby allowing future probes to be hybridized without denaturing the primary oligonucleotides from their targets. In some embodiments, step (e) of this method involves registering the images produced by the method. As such, in some embodiments, the method further includes registering the images produced in step (h).

In these embodiments, there are binding sites for at least two (e.g., 4-20 or sometimes more) different probes in the tails of the primary oligonucleotides. The identities of the probes that hybridize to a particular site on the RNA blot identifies the primary probe that is hybridized to that site, which, in turn, allows one to determine which species of RNA is immobilized at that site (i.e., by its gene name). The following hypothetical example shows how a total of 7 species of RNAs (RNAs 1-7) that are tethered to different sites of a blot can be identified using seven sets of unlabeled primary oligonucleotides (POs 1-7) that hybridize to the RNAs, and three labeled probes (A, B and C), where the tails of the primary oligonucleotides have binding sites for A, B or C, or any combination thereof.

Example Lookup Table

| RNA name (detected features) | Code | Probe A | Probe B | Probe C |
|---|---|---|---|---|
| RNA/PO 1 (A only) | 100 | Yes | No | No |
| RNA/PO 2 (B only) | 010 | No | Yes | No |
| RNA/PO 3 (C only) | 001 | No | No | Yes |
| RNA/PO 4 (A and B) | 110 | Yes | Yes | No |
| RNA/PO 5 (B and C) | 011 | No | Yes | Yes |
| RNA/PO 6 (A and C) | 101 | Yes | No | Yes |
| RNA/PO 7 (A, B and C) | 111 | Yes | Yes | Yes |

In this hypothetical experiment, probes A, B and C are hybridized in order and the RNAs that the probes hybridize to can be identified by the codes, where 1 indicates that the probe hybridizes and 0 indicates that the probe does not hybridize. So, if a particular position on the blot only binds to probes A and C, then the RNA at that position is RNA 6 (which, in many cases, would be encoded by Gene 6). Examples of such encoding methods, including error-corrected versions of the same, can be adapted from, e.g., Moffitt et al (Methods Enzymol. 2016 572: 1-49) and Moffit et al (Proc. Natl. Acad. Sci. 2016 113: 11046-51).

Using the same method, 15 genes can be distinguished by 4 probes, 31, 15 genes can be distinguished by 4 probes, 32 genes can be distinguished by 5 probes, 31 genes can be distinguished by 5 probes, 63 genes can be distinguished by 6 probes, 127 genes can be distinguished by 6 probes, 255 genes can be distinguished by 7 probes, and so on. If the probes have distinguishable labels, multiple probes can potentially be hybridized in a single cycle, meaning that in theory, several hundred species of RNA could be identified in as few as three cycles.

In some embodiments, a probabilistic barcode caller is used for identifying a nucleic acid (e.g., RNA) from the detected signals (e.g., detected by using the probes). The probabilistic barcode caller is useful because it can utilize the intensity information. Compared to a simple threshold-based method, which determines the detection or presence of a particular probe based on the intensity for the particular probe exceeding a threshold, the probabilistic barcode caller utilizes the intensity information for a particular prober in conjunction with the intensity information for one or more other probes so that it can reduce erroneous identification of the nucleic acid. In addition, the probabilistic barcode caller can identify a corresponding nucleic acid by probabilistic mapping of the detected signals to one of nucleic acid molecules in a pool of candidate nucleic acid molecules (or to one of codes in a pool of candidate codes), which eliminates the need for (i) side-by-side comparison or matching of the detected code against valid codes in a codebook or (ii) an error detection or correction system.

After reading the blot in each cycle, in some embodiments, the method includes inactivating or removing the labels that are associated with (i.e., hybridized to) the blot, leaving the primary oligonucleotides still bound to the RNA. The labels that are associated with the blot may be removed or inactivated by a variety of methods including, but not limited to, denaturation (in which case the label and the probe in its entirety may be released and can be washed away), by cleaving a linkage in the probe (in which case the label and part of the probe may be released and can be washed away), by cleaving both the probe and the oligonucleotide to which the probe is hybridized (to release a fragment that can be washed away), by cleaving the linkage between the probe and the label (in which case the label will be released and can be washed away and can be washed away), or by inactivating the label itself (e.g., by breaking a bond in the label, thereby preventing the label from producing a signal). In all of these removal methods, the primary oligonucleotides that are hybridized to the RNA are left intact and free to hybridize to labeled probes in future (or subsequent) cycles. In some embodiments, fluorescence may be inactivated by peroxide-based bleaching or cleavage of a fluorophore linked to a nucleotide through a cleavable linker (e.g., using TCEP as a cleaving reagent).

In some embodiments, each of labeled probes includes: an oligonucleotide and a label, and the oligonucleotide and the label are connected to each other via a cleavable linker. If the labeled probes contain a cleavable linker, then the cleavable linker should be capable of being selectively cleaved using a stimulus (e.g., a chemical, light or a change in its environment) without breaking any bonds in the oligonucleotides. In some embodiments, the cleavable linkage may be a disulfide bond, which can be readily broken using a reducing agent (e.g., β-mercaptoethanol, TCEP or the like). Suitable cleavable bonds that may be employed include, but are not limited to, the following: base-cleavable sites such as esters, particularly succinates (cleavable by, for example, ammonia or trimethylamine),f quaternary ammonium salts (cleavable by, for example, diisopropylamine) and urethanes (cleavable by aqueous sodium hydroxide); acid-cleavable sites such as benzyl alcohol derivatives (cleavable using trifluoroacetic acid), teicoplanin aglycone (cleavable by trifluoroacetic acid followed by base), acetals and thioacetals (also cleavable by trifluoroacetic acid), thioethers (cleavable, for example, by HF or cresol) and sulfonyls (cleavable by trifluoromethane sulfonic acid, trifluoroacetic acid, thioanisole, or the like); nucleophile-cleavable sites such as phthalamide (cleavable by substituted hydrazines), esters (cleavable by, for example, aluminum trichloride); and Weinreb amide (cleavable by lithium aluminum hydride); and other types of chemically cleavable sites, including phosphorothioate (cleavable by silver or mercuric ions) and diisopropyldialkoxysilyl (cleavable by fluoride ions). Other cleavable bonds will be apparent to those skilled in the art or are described in the pertinent literature and texts (e.g., Brown (1997) Contemporary Organic Synthesis 4(3); 216-237). In some embodiments, a cleavable bond may be cleaved by an enzyme. In particular embodiments, a photocleavable (“PC”) linker (e.g., a UV-cleavable linker) may be employed. Suitable photocleavable linkers for use may include ortho-nitrobenzyl-based linkers, phenacyl linkers, alkoxybenzoin linkers, chromium arene complex linkers, NpSSMpact linkers and pivaloylglycol linkers, as described in Guillier et al. (Chem Rev. 2000 Jun. 14; 100(6):2091-158). Exemplary linking groups that may be employed in the subject methods may be described in Guillier et al., supra and Olejnik et al. (Methods in Enzymology 1998 291:135-154), and further described in U.S. Pat. No. 6,027,890; Olejnik et al. (Proc. Natl. Acad Sci, 92:7590-94); Ogata et al. (Anal. Chem. 2002 74:4702-4708); Bal et al. (Nucl. Acids Res. 2004 32:535-541); Zhao et al. (Anal. Chem. 2002 74:4259-4268); and Sanford et al. (Chem Mater. 1998 10:1510-20), and are purchasable from Ambergen (Boston, MA; NHS-PC-LC-Biotin), Link Technologies (Bellshill, Scotland), Fisher Scientific (Pittsburgh, PA) and Calbiochem-Novabiochem Corp. (La Jolla, CA).

In some embodiments, the cleavable linker may comprise a linkage cleavable by a reducing agent (e.g., a disulfide bond). In these embodiments, the label may be removed using a reducing agent, e.g., tris(2-carboxyethyl)phosphine (TCEP).

As described above, in some embodiments, each cycle of the method uses a single labeled probe, or at least 2 labeled probes. (e.g., 2, 3 or 4 labeled probes). In the latter embodiments, the probes may be distinguishably labeled so that their signals can be separately detected. Suitable distinguishable fluorescent label pairs useful in the subject methods include Cy-3 and Cy-5 (Amersham Inc., Piscataway, NJ), Quasar 570 and Quasar 670 (Biosearch Technology, Novato CA), Alexafluor555 and Alexafluor647 (Molecular Probes, Eugene, OR), BODIPY V-1002 and BODIPY V1005 (Molecular Probes, Eugene, OR), POPO-3 and TOTO-3 (Molecular Probes, Eugene, OR), and POPRO3 and TOPRO3 (Molecular Probes, Eugene, OR). Further suitable distinguishable detectable labels may be found in Kricka et al. (Ann Clin Biochem. 39:114-29, 2002), Ried et al. (Proc. Natl. Acad. Sci. 1992: 89: 1388-1392) and Tanke et al. (Eur. J. Hum. Genet. 1999 7:2-11) and others. In some embodiments three or four distinguishable dyes may be used. Specific fluorescent dyes of interest include: xanthene dyes, e.g., fluorescein and rhodamine dyes, such as fluorescein isothiocyanate (FITC), 6-carboxyfluorescein (commonly known by the abbreviations FAM and F), 6-carboxy-2′,4′,7′,4,7-hexachlorofluorescein (HEX), 6-carboxy-4′, 5′-dichloro-2′, 7′-dimethoxyfluorescein (JOE or J), N,N,N′,N′-tetramethyl-6-carboxyrhodamine (TAMRA or T), 6-carboxy-X-rhodamine (ROX or R), 5-carboxyrhodamine-6G (R6G⁵ or G⁵), 6-carboxyrhodamine-6G (R6G⁶ or G⁶), and rhodamine 110; cyanine dyes, e.g., Cy3, Cy5 and Cy7 dyes; coumarins, e.g., umbelliferone; benzimide dyes, e.g. Hoechst 33258; phenanthridine dyes, e.g., Texas Red; ethidium dyes; acridine dyes; carbazole dyes; phenoxazine dyes; porphyrin dyes; polymethine dyes, e.g., BODIPY dyes and quinoline dyes. Specific fluorophores of interest that are commonly used in subject applications include: Pyrene, Coumarin, Diethylaminocoumarin, FAM, Fluorescein Chlorotriazinyl, Fluorescein, R110, Eosin, JOE, R6G, Tetramethylrhodamine, TAMRA, Lissamine, Napthofluorescein, Texas Red, Cy3, and Cy5, etc. As noted above, within each subset of probes, the fluorophores may be chosen so that they are distinguishable, i.e., independently detectable, from one another, meaning that the labels can be independently detected and measured, even when the labels are mixed. In other words, the amounts of label present (e.g., the amount of fluorescence) for each of the labels are separately determinable, even when the labels are co-located (e.g., in the same tube or in the same area of the section).

In any embodiment, particularly those in which the RNA has been transferred to the support by electrophoresis and therefore the support has an electrically conductive surface, hybridization of the primary oligonucleotide probes and/or labeled probes may be assisted by electrophoresis. In some embodiments, electrophoresis is used to concentrate the probes at the surface of the substrate, thereby increasing the probability that they will hybridize to their targets.

In some embodiment, as noted below, the primary oligonucleotide probes may be made by reverse-transcribing RNA that has been made from a mixed PCR product via IVT. In these embodiments, the reverse transcription primer may have an added group (a “crosslinking-moiety”) that will become part of the primary oligonucleotide probes and can be used to affix the primary oligonucleotide probes to the substrate. In one example, the primary oligonucleotide probes may be amine modified (e.g., at or within 30 bases of the 5′ end) and, after they are hybridized to the RNA they can be affixed to the substrate by treatment with paraformaldehyde (PFA). In these embodiments, the substrate may be coated in poly-L-lysine, or another coating that can be linked to the amine modification using a crosslinker. In these embodiments, the method may comprise transferring the RNA onto a coated substrate (e.g., that is coated in poly-L-lysine) to produce the blot, removing the substrate, crosslinking the RNA to the substrate, e.g., using PFA, hybridizing the RNA blot with a set of unlabeled primary oligonucleotides that contain a crosslinking-moiety and that that hybridize to different sites in the same RNA species and have a tail, crosslinking the unlabeled primary oligonucleotides to the substrate (such that the crosslinking-moiety moiety of the unlabeled primary oligonucleotides becomes covalently linked to the poly-L-lysine or to other groups that are on the surface of the substrate, such as residual formaldehyde or the RNA), e.g., using PFA, and then hybridizing the blot with the labeled probes, as described above. In some embodiments, the blot or substrate may comprise one or more fiducial markings that can be used for image registration. In these embodiments, the fiducial markings may be fluorescent beads that are deposited on the substrate. In addition, the method may further comprise staining and imaging the sample prior to its removal from the substrate. For example, in addition to the labeling methods described above, the sample may be stained using a cytological stain, either before or after performing the method described above. In these embodiments, the stain may be, for example, phalloidin, gadodiamide, acridine orange, bismarck brown, barmine, Coomassie blue, bresyl violet, brystal violet, DAPI, hematoxylin, eosin, ethidium bromide, acid fuchsine, haematoxylin, hoechst stains, iodine, malachite green, methyl green, methylene blue, neutral red, Nile blue, Nile red, osmium tetroxide (formal name: osmium tetraoxide), rhodamine, safranin, phosphotungstic acid, osmium tetroxide, ruthenium tetroxide, ammonium molybdate, cadmium iodide, carbohydrazide, ferric chloride, hexamine, indium trichloride, lanthanum nitrate, lead acetate, lead citrate, lead(II) nitrate, periodic acid, phosphomolybdic acid, potassium ferricyanide, potassium ferrocyanide, ruthenium red, silver nitrate, silver proteinate, sodium chloroaurate, thallium nitrate, thiosemicarbazide, uranyl acetate, uranyl nitrate, vanadyl sulfate, or any derivative thereof. The stain may be specific for any feature of interest, such as a protein or class of proteins, phospholipids, DNA (e.g., dsDNA, ssDNA), RNA, an organelle (e.g., cell membrane, mitochondria, endoplasmic recticulum, golgi body, nuclear envelope, and so forth), or a compartment of the cell (e.g., cytosol, nuclear fraction, and so forth). The stain may enhance contrast or imaging of intracellular or extracellular structures. In some embodiments, the sample may be stained with DAPI or haematoxylin and eosin (H&E).

In any embodiment, each set of primary oligonucleotides may comprise at least 1, at least 5, or at least 10 (e.g., 10-100) primary oligonucleotides. Within each set, the primary oligonucleotides hybridize to the same species of RNA, i.e., transcripts encoded by the same gene. The primary oligonucleotides of the different sets hybridize to different RNA species (i.e., transcripts encoded by different genes). Therefore, each RNA species that is going to be examined will typically be targeted by a single set of primary oligonucleotides. As such, the number of sets of unlabeled primary oligonucleotides used in the method generally depends on how many RNA species are going to be examined. As noted above, the method can potentially be used to examined hundreds or thousands of RNA species. As such, in some cases, at least 10 sets (e.g., 10-5000 sets) of unlabeled primary oligonucleotides may be used, where each set comprises 10-100 unlabeled primary oligonucleotides.

As would be apparent, the sequences of the oligonucleotides used may be selected in order to minimize background staining, either from non-specific adsorption, through binding to endogenous genomic sequences (RNA or DNA) or cross-hybridization to one another (except for when it is desirable). Likewise, the hybridization and washing buffers may be designed to minimize background staining either from non-specific adsorption or through binding to endogenous genomic sequences (RNA or DNA) or through binding to other reporter sequences. As would be apparent, the primary oligonucleotides and probes should be designed so that they are orthogonal in that they only bind to their desired target and not to other primary oligonucleotides or probes. In some embodiments, the labeled probes may have a calculated Tm in the range of 15° C. to 70° C. (e.g., 20° C.-60° C. or 35° C.-50° C.) such that the duplexes of the hybridization step have a Tm that lower (e.g., at least 10° C. or at least 20° C. lower than the Tm of the RNA target binding sequence of the primary oligonucleotides. In addition, it is well known that DNA/RNA duplexes have a higher melting temperature than the DNA/DNA duplexes having the same sequence. In practice, the primary oligonucleotide probes have a longer binding region than the labeled probes and, in some embodiment, the primary oligonucleotide probes may be attached to the substrate. The labeled probes are hybridized and washed at a temperature that is well below the melting temperature of the primary oligonucleotide probes. In some embodiments, the labeled nucleic acid probes may be 8 to 25 nucleotides in length, e.g., 10 to 18 nucleotides or 11 to 17 nucleotides in length although, in some embodiments, the probe may be as short as 5 nucleotides in length to as long as 150 nucleotides in length (e.g., 6 nucleotides in length to 100 nucleotides in length). In some embodiments, the primary oligonucleotides have two sections, a first section of 20-nucleotides that hybridizes to an RNA, and one or more second sections of 20-150 nucleotides (e.g., 15-100 nucleotides) that do not hybridize to an RNA and provide a binding site for one or more labeled probes (which binding sites are not overlapping) in the primary oligonucleotides. In some embodiments, the primary oligonucleotides may comprise at least three sections. In these embodiments, the oligonucleotides may contain a first section, as discussed above, flanked a second section and a third section, where the second and third sections are 5′ and 3′ to the first section and provide binding sites for the labeled probes.

In this method, the blot may be read using any convenient reading method and, in some embodiments, the blot can be read using a fluorescence microscope equipped with an appropriate filter for each fluorophore, or by using dual or triple band-pass filter sets to observe multiple fluorophores (see, e.g., U.S. Pat. No. 5,776,688). In some embodiments, each reading step produces an image of the blot showing the pattern of binding of a subset of probes. In these embodiments, the images may be registered, and each corresponding spot of the images may be analyzed using the method described above to identify which RNA species is at spot. The image analysis module used may transform each RNA species into a different color to produce a false color images of the blot, where the colors indicate the different RNA species. The image analysis module may further be configured to adjust (e.g., normalize) the intensity and/or contrast of signal intensities or false colors, to perform a convolution operation (such as blurring or sharpening of the intensities or false colors), or perform any other suitable operations to enhance the image. The image analysis module may perform any of the above operations to align pixels obtained from successive images and/or to blur or smooth intensities or false colors across pixels obtained from successive images.

As noted above, since the blot is already planar, there is no need for images to be taken through a tissue in different focal planes, in the z direction. In some embodiments, the surface of the substrate may be uneven or slightly curved because of how the substrate is mounted in the flow cell/microscope. In these embodiments, a reduced z-stack may still be taken. In other embodiments, the substrate may be held flat which effectively renders the z-stack unnecessary, particularly if the microscope can detect the surface of the substrate.

As would be apparent, the image analysis method may be implemented on a computer. In certain embodiments, a general-purpose computer can be configured to a functional arrangement for the methods and programs disclosed herein. The hardware architecture of such a computer is well known by a person skilled in the art, and can comprise hardware components including one or more processors (CPU), a random-access memory (RAM), a read-only memory (ROM), an internal or external data storage medium (e.g., hard disk drive). A computer system can also comprise one or more graphic boards for processing and outputting graphical information to display means. The above components can be suitably interconnected via a bus inside the computer. The computer can further comprise suitable interfaces for communicating with general-purpose external components such as a monitor, keyboard, mouse, network, etc. In some embodiments, the computer can be capable of parallel processing or can be part of a network configured for parallel or distributive computing to increase the processing power for the present methods and programs. In some embodiments, the program code read out from the storage medium can be written into a memory provided in an expanded board inserted in the computer, or an expanded unit connected to the computer, and a CPU or the like provided in the expanded board or expanded unit can actually perform a part or all of the operations according to the instructions of the program code, so as to accomplish the functions described below. In other embodiments, the method can be performed using a cloud computing system. In these embodiments, the data files and the programming can be exported to a cloud computer, which runs the program, and returns an output to the user.

Systems for analyzing a sample are also provided. In some embodiments, the subject systems include an electrophoresis device, as described above and illustrated in FIG. 1A, as well as a slide-compatible liquid-handling workstation that is capable of performing the method as described above, and a microscope.

In some embodiments, a gel could be cast into the tissue the molecules of interest could be crosslinked in the gel via a cleavable crosslinker. After the molecules are attached to the gel, the tissue could be digested away so that only the gel and molecule of interest remain. After this step, the crosslinker could be cleaved to release the molecules of interest and electrophoresis could be used to move them to the planar substate. The gel could be polyacrylamide or agarose or similar.

In some embodiments, a thin spacer, e.g., a gel, could be placed between the capture slide and the tissue.

Further, to detect proteins, one could pre-stain the sample using antibodies that are tagged with a cleavable oligonucleotide barcode, which could be cleaved off and moved onto the substrate at the same time as the RNA. Fluorescence in situ hybridization (FISH) could be used to detect these barcodes and thus indirectly measure protein content and localization.

In alternative embodiments, the RNA could be first reverse transcribed and the cDNA could be captured. This might be advantageous in samples were the RNA is heavily fixed like in Formalin Fixed Paraffin Embedded (FFPE) samples.

The methods described herein find general use in a wide variety of applications for analysis of any sample (e.g., in the analysis of tissue sections, sheets of cells, spun-down cells, etc.). Further, the method has a variety of clinical applications, including, but not limited to, diagnostics, prognostics, disease stratification, personalized medicine, clinical trials and drug accompanying tests.

In particular embodiments, the sample may be a section of any tissue, including skin (melanomas, carcinomas, etc.), soft tissue, bone, breast, colon, liver, kidney, adrenal, gastrointestinal, pancreatic, gall bladder, salivary gland, cervical, ovary, uterus, testis, prostate, lung, thymus, thyroid, parathyroid, pituitary (adenomas, etc.), brain, spinal cord, ocular, nerve, and skeletal muscle, etc. In some embodiments, the sample may be a tissue biopsy obtained from a patient. Biopsies of interest include both tumor and non-neoplastic biopsies of any tissue.

In any embodiment, data can be forwarded to a “remote location”, where “remote location,” means a location other than the location at which the image is examined. For example, a remote location could be another location (e.g., office, lab, etc.) in the same city, another location in a different city, another location in a different state, another location in a different country, etc. As such, when one item is indicated as being “remote” from another, what is meant is that the two items can be in the same room but separated, or at least in different rooms or different buildings, and can be at least one mile, ten miles, or at least one hundred miles apart. “Communicating” information refers to transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. Examples of communicating media include radio or infra-red transmission channels as well as a network connection to another computer or networked device, and the internet or including email transmissions and information recorded on websites and the like. In certain embodiments, the image may be analyzed by an MD or other qualified medical professional, and a report based on the results of the analysis of the image may be forwarded to the patient from which the sample was obtained.

In certain embodiments, two different samples may be compared using the above methods. The different samples may be composed of an “experimental” sample, i.e., a sample of interest, and a “control” sample to which the experimental sample may be compared. In many embodiments, the different samples are pairs of cell types or fractions thereof, one cell type being a cell type of interest, e.g., an abnormal cell, and the other a control, e.g., normal, cell. If two fractions of cells are compared, the fractions are usually the same fraction from each of the two cells. In certain embodiments, however, two fractions of the same cell may be compared. Exemplary cell type pairs include, for example, cells isolated from a tissue biopsy (e.g., from a tissue having a disease such as colon, breast, prostate, lung, skin cancer, or infected with a pathogen, etc.) and normal cells from the same tissue, usually from the same patient; cells grown in tissue culture that are immortal (e.g., cells with a proliferative mutation or an immortalizing transgene), infected with a pathogen, or treated (e.g., with environmental or chemical agents such as peptides, hormones, altered temperature, growth condition, physical stress, cellular transformation, etc.), and a normal cell (e.g., a cell that is otherwise identical to the experimental cell except that it is not immortal, infected, or treated, etc.); a cell isolated from a mammal with a cancer, a disease, a geriatric mammal, or a mammal exposed to a condition, and a cell from a mammal of the same species, preferably from the same family, that is healthy or young; and differentiated cells and non-differentiated cells from the same mammal (e.g., one cell being the progenitor of the other in a mammal, for example). In one embodiment, cells of different types, e.g., neuronal and non-neuronal cells, or cells of different status (e.g., before and after a stimulus on the cells) may be employed. In another embodiment of the invention, the experimental material contains cells that are susceptible to infection by a pathogen such as a virus, e.g., human immunodeficiency virus (HIV), etc., and the control material contains cells that are resistant to infection by the pathogen. In another embodiment, the sample pair is represented by undifferentiated cells, e.g., stem cells, and differentiated cells.

The images produced by the method may be viewed side-by-side or, in some embodiments, the images may be superimposed or combined. In some cases, the images may be in color, where the colors used in the images may correspond to the labels used.

Cells from any organism, e.g., from bacteria, yeast, plants and animals, such as fish, birds, reptiles, amphibians and mammals may be used in the subject methods. In certain embodiments, mammalian cells, i.e., cells from mice, rabbits, primates, or humans, or cultured derivatives thereof, may be used.

### EXAMPLES

The following examples illustrate procedures for practicing some embodiments of the invention. These examples should not be construed as limiting. All percentages are by weight and all solvent mixture proportions are by volume unless otherwise noted.

### Materials and Methods

The following description describes an embodiment of the present method called Enhanced ELectric (“EEL”) FISH, in which the RNAs are detected by multiple rounds of labeling, imaging and label removal. In brief, the EEL method includes three parts. First, the capture slide is produced, and a tissue slice is positioned on top of it. Second, the RNA is transferred to the capture slide and the tissue is removed. Third, the captured RNA molecules are hybridized with probes that encode a pre-defined barcode and this barcode is read by multiple cycles of labeling, imaging and label removal.

Capture Slide

The capture slide is designed to be conductive, optically transparent and to efficiently capture RNA molecules.

As a conductive substrate glass that is coated with a thin layer of Indium Tin-oxide (ITO) was used. ITO coated glass has a high transmission coefficient for visible light, enabling its use in microscopy, while also being conductive to serve as the positively charged anode attracting the negatively charged RNA molecule cations.

In this example, although it is not necessary, to immobilize the RNA molecules on the capture surface, the ITO surface was modified to contain poly-T oligonucleotides that can hybridize to the poly-A tail of mRNA molecules. Additionally, poly-D-lysine was added to the surface that, because of its amino groups, is positively charged, attracting the negatively charged RNA. These steps are thought to assist in immobilizing the RNA on the surface by electrostatic attraction and provides a chemical substrate to fixate the RNA molecules to the surface with formaldehyde (PFA). Fluorescent beads were also deposited on the surface to facilitate the alignment and stitching of the images.

Once the surface has prepared, a thin tissue section was placed on top of the surface.

RNA Transfer

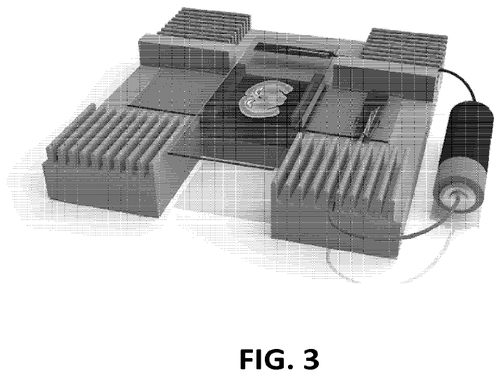

To facilitate the movement of the RNA through the tissue, the cell membranes were permeabilized using a surfactant (in the case, Triton X-100), and the protein was denatured using a reducing agent (in this case, dithiothreitol (DTT)). The electrophoreses setup is shown in FIG. 3. The electrophoresis setup includes a sandwich of the capture slide with the tissue sample facing upwards, and a top electrode with two spacers in between (FIG. 1B). In this example, the top electrode is also made of an ITO coated glass slides, but can also be a gold electrode or other electrically conductive metals or chemicals. Wires are attached to the conductive surfaces by means of conductive copper tape and these are attached to a power source. The electric potential applied to the setup is 15 V/cm. 1-mm spacers were used, so the applied voltage is 1.5 V. The conductive liquid in which the electrophoresis is performed in is Tris Buffered EDTA (TBE) with the addition of DTT and urea to denature the RNA and any other proteins and other biomolecules.

After the electrophoresis is performed, the tissue was removed by digesting the proteins with Proteinase K and washing in sodium dodecyl sulfate (SDS). Then, the captured RNA molecules were fixed to the capture slide with paraformaldehyde (PFA) and the encoding probes were hybridized to the RNA molecules that are tethered to slide.

Detection

In this example, the encoding probes contain a specific RNA binding sequence that hybridizes to the RNA, and one or two tails that contain multiple sequences for the barcode detection. For the set of genes, a binary code set is pre-determined, and each gene is assigned a specific barcode. For example, with a 5-bit barcode, gene A could have the barcode 10011, which means that an RNA molecule of gene A will be labeled in round 1, 4 and 5. After one bit of the barcode is labeled and imaged, the fluorophore of the detection oligonucleotide is cleaved off by reduction of the thiol linker with the reducing agent Tris(2-carboxyethyl)phosphine (TCEP).

All of the detection steps can be performed in a fluidic system and integrated with microscope.

Further details of this implementation of the method are described below.

Method details Glass cleaning: ITO coated 24 mm×60 mm coverslips were cleaned by sonicating them in acetone, then 2-propanol and then dH₂O for 20 minutes, and then stored in dH₂O. Optional to also perform plasma cleaning.

Glass functionalization: After drying, the slides were submerged in a 2% solution of (3-Glycidyloxypropyl)Trimethoxysilane (GPTMS) in anhydrous acetone under a nitrogen gas atmosphere for 2 hours, followed by one wash with acetone and then dried using nitrogen gas. The ITO side of the glass was identified by measuring conductivity with a multimeter. 40 ul of spotting solution containing 10 μM poly-T 60 modified on the 3′ end with an amine group and 1× Schott spotting solution of epoxy-amino coupling, was added to the slide and covered with a plastic coverslip. The slides were incubated 1 hour at 25° C. to allow for the amine groups to bind the epoxy groups of the GPTMS. Slides were washed 5 times with 2× sodium chloride sodium citrate (SSC) buffer. Then remaining epoxide groups were blocked by poly-D-lysine (molecular weight 70.000-150.000) for 30 minutes in water at room temperature, followed by 3 washes with dH₂O. Then fluorescent beads were deposited on the surface by placing 100 μl of 0.006% solids in dH₂O on the surface for 3 minutes. The bead solution was removed, and slides were air dried.

Tissue capture: A 10 μm cryosection was cut from a fresh frozen tissue sample and placed on the functionalized slide.

RNA transfer: The tissue slice can be optionally imaged before starting the RNA transfer. For example, the nuclei may be imaging after staining with Hoechst or DAPI.

The tissue was permeabilized on the slide for 5 minutes with 0.1% Triton X-100 and mM DTT in 1×TBE buffer, followed by 5 washes with TBE. A wire was attached to the capture slide with a piece of copper tape with conductive glue and the capture slide was placed in the electrophoresis setup. Then two 1-mm thick spacers made of PDMS are positions on either side of the tissue slice and a top electrode made of ITO coated glass and wire is positioned on top. Electrophoresis buffer comprised of 10 mM DTT, 1M urea in 1× TBE buffer was injected into the space and 1.5 V was applied to the electrodes, where the positive pole is attached to the capture slide. RNA can also be transferred in water. After 20 minutes of electrophoresis the setup was disassembled and the tissue was digested by 3 washes of 10 minutes in 2.4 U/ml Proteinase K (NEB), 1% SDS, 20 mM Tris HCL pH 7.4 and 5 ul/ml Superase (Thermo) RNase inhibitor at 30° C. The concentration of proteinase K and incubation time can be experimentally adjusted to the type of sample. Then the remaining tissue residue was washed away with 5% SDS in 2×SSC (3 washes for 5 minutes at 30° C.). After 5 further washes with 2×SSC the RNA was crosslinked to the surface by fixing 10 minutes with 4% PFA in 1×PBS buffer, followed by 3 washes with 1×PBS and 3 washes with 2×SSC.

Encoding probes were dissolved to a final concentration of 1 nM/probe in hybridization mix containing 30% formamide, 0.1 g/ml Dextran sulfate, 1 mg/ml E. Coli tRNA, 2 mM Ribonucleoside Vanadyl complexes (RVC) and 2×SSC. A drop of 20 μl was placed on the capture slide and covered with a cover slip and hybridized for 48 hours at 38.5° C.

RNA detection: The capture slide was placed in a heat-controlled flow cell, connected to an automated fluidic system and placed on the microscope. Unbound probes were removed by 4 washes with 30% formamide in 2×SSC for 15 minutes at 47° C. Then fluorescent detection probes that bind to bit 1 are introduced in hybridization mix with a concentration of nM/probe and hybridized in 10 minutes at 37° C. Unbound probes were washed away with 20% formamide in 2×SSC by 3 washes for 2 minutes followed by 5 washes of SSC 2×. Then, imaging buffer containing, 2 mM TROLOX, 5 mM 3,4-dihydroxybenzoic acid, 20 nM protocatechuate-3,4-dioxygenase in 2×SSC was injected into the flow cell. Imaging was performed with an automated Nikon Ti2 microscope. Afterwards the fluorophores attached by a thiol linker are cleaved off the detection probe by two washes of 10 minutes with 50 mM TCEP in 2×SSC at 20° C. After 10 washes with 2×SSC, the next detection probe for bit 2 is introduced. From here on, labeling—detection and stripping cycles are performed until all beads of the barcode are detected.

### Results

This method is routinely performed on mouse brain, human adult brain and human developing head, with good results. Below are a number of examples demonstrating that the method works.

### Example 1

### Large Scale Anatomy

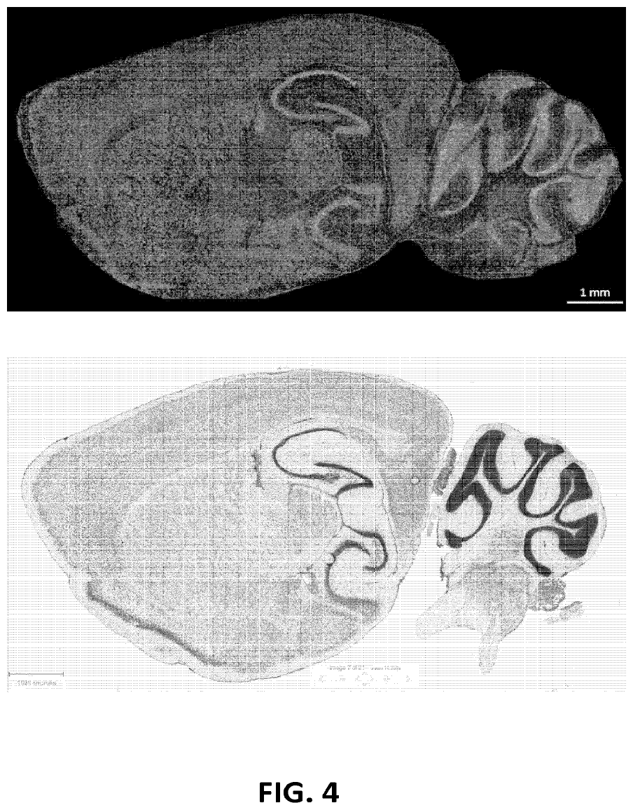

The protocol outlined above was used to examine RNA expressed by 167 genes in a mouse brain section that was cut in the sagittal orientation. FIG. 4 shows exemplary results. Top panel: every colored spot in this figure is a single molecule that was detected, where the different colors correspond to the 167 genes. Due to the high density in dots, it is hard to see individual dots but what can be seen is that the signal distribution matches the anatomical structure of the mouse brain. To verify this, the picture in the bottom panel comes from the anatomical mouse brain atlas generated by the Allen Institute. The picture in the bottom panel is obtained by using a Nissl staining which labels neurons in the brain and shows the general cell densities. As expected, more signals are detected in areas where there are more cells, confirming that anatomical features are accurately detected.

### Example 2

### Gene Expression Patterns

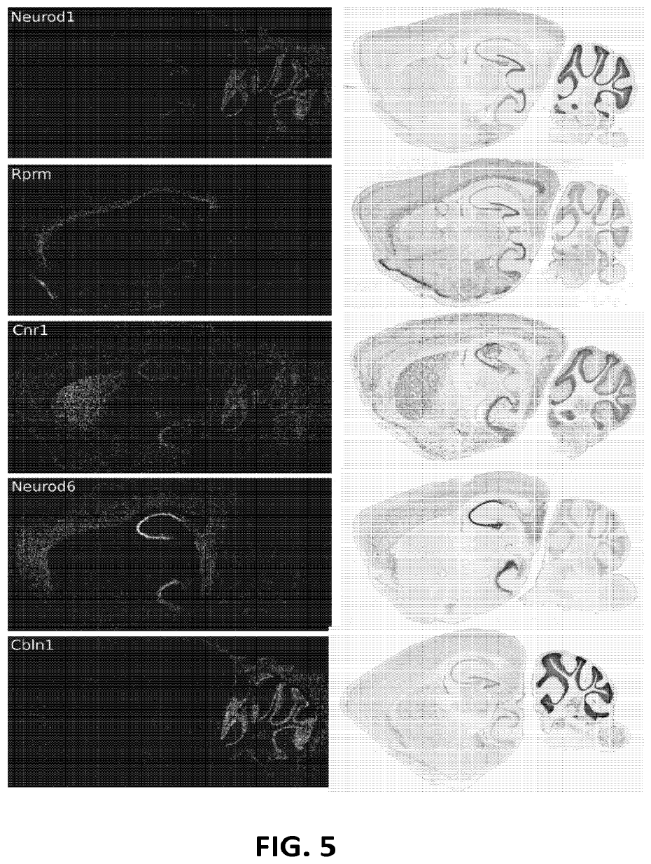

To confirm that the detected signals shown in the top panel FIG. 4 is also anatomically correct, it was compared to the Allen brain expression atlas. The Allen Atlas contains in situ Hybridization staining's for single genes in the mouse brain and is regarded as a standard in the field for gene expression localization. Five comparisons are shown in FIG. 5, where data obtained by the method described herein is shown on the left and the Allen Atlas results are shown on the right. Each gene accurately matches the known expression pattern in the brain.

### Example 3

### Single Cell Resolution

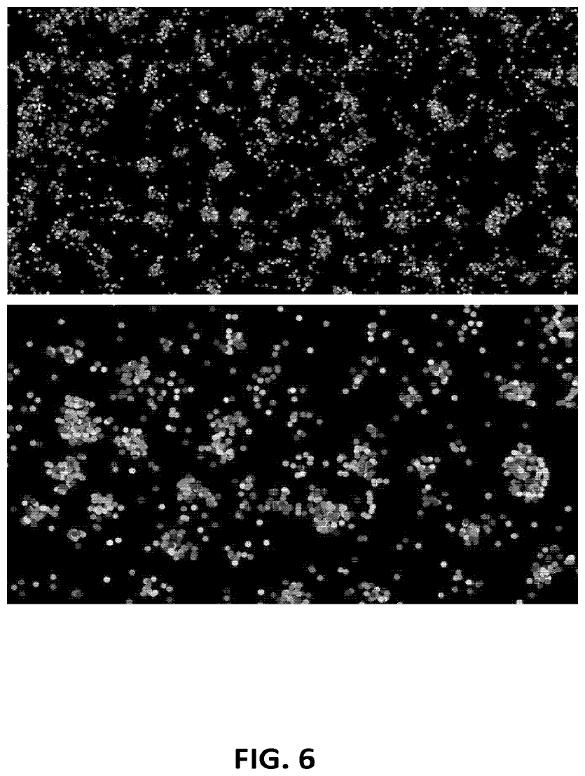

Zooming in into the images reveals that the signal is clustered which is what is expected of signal coming from individual cells. In the brain, cells are sparsely distributed, and cell bodies are not touching (except for regions in the hippocampus and cerebellum). Furthermore, most RNA is located in the cell body and therefore a clustered RNA pattern coming from the cell bodies is expected. The images shown in FIG. 6 are examples of the first experiment with 167 genes in the mouse brain that shows this clustered pattern, indicating that RNA comes from individual cells placed above. Again, each dot is one detected molecule, and each color corresponds to (or represents or indicates) one of the 167 genes.

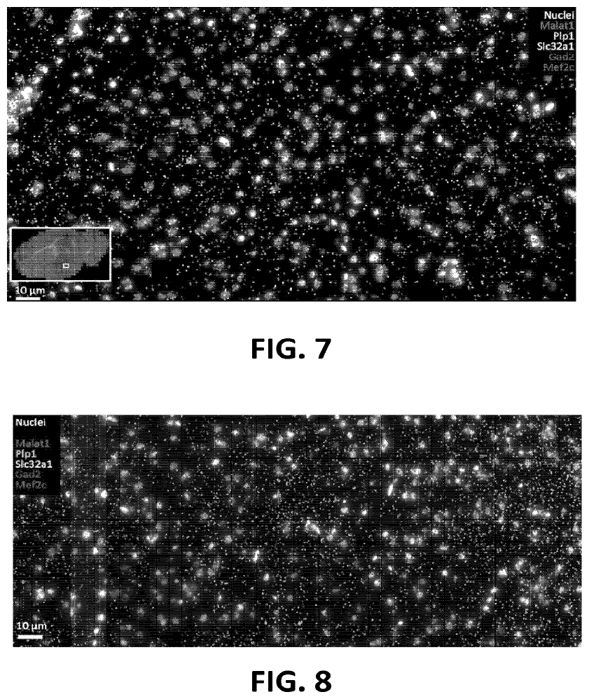

To confirm this, the detected expression pattern was overlaid with images taken form the nuclei of each cell before-hand. In FIG. 7, the white color is the image of the nuclei and the dots indicate molecules of the 6 genes. It can be seen that most dots seem to be close to a nucleus of a cell, confirming that with the disclosed method, the RNA is transferred accurately and maintains single-cell resolution.

### Example 4

### Effect of Electrophoresis

In contrast, if the same experiment as described above is repeated but without applying the electric field and the electrostatic attraction, the molecules seem to diffuse more and do not match the cellular patter observed with the electrophoresis. FIG. 8 shows an image of a matched sample as above but without the electric force, where it is clear that the dots are not close to the nuclei. This suggest that the electrical field may be needed to get an anatomically correct print.

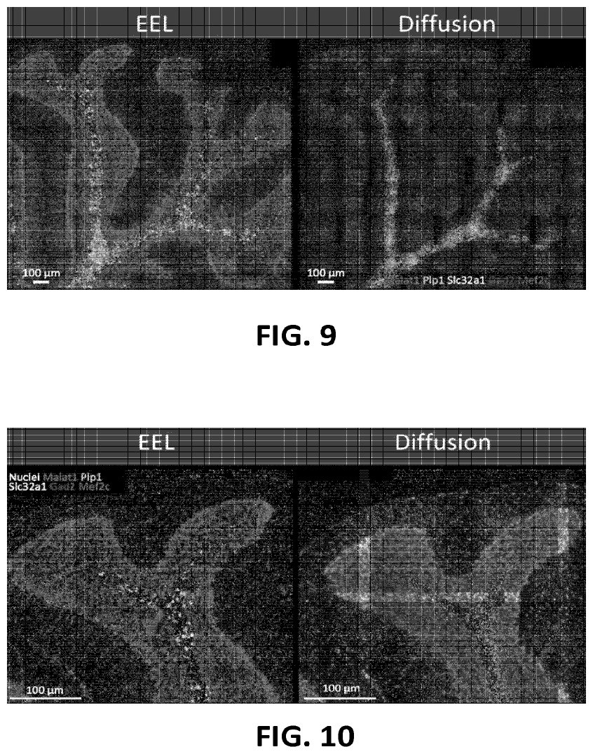

This difference can also be seen on a larger scale. FIG. 9 shows a comparison with and without the electric force (e.g., electrophoresis) applied to the cerebellum of the brain. In FIG. 9, it is clear that the electric force is important to increase the efficiency of the transfer, as there are more detected molecules in the electric force (e.g., EEL) condition as opposed to just diffusion.

Furthermore, the anatomy seems distorted in the diffusion condition. FIG. 10 shows a zoom-in of the above-described experiment, but now also overlaid with the nuclei image. On the left, in the electrophoresis treatment, most of the detected molecules are in an area where there are a lot of cells. In fact, the image of the nuclei underneath (shown in white) is hard to see because there are so many detected molecules. On the right-side image, the nuclei can be seen, and most of the signal is located outside the area with a high cellular density, indicating that just diffusion of the molecules distorts the tissue blot.

The image shown in FIGS. 7-10 were obtained using slides that were coated in poly-D-lysine.

### Example 5

### Speed

In using this method, the speed at which one can process tissue samples is much increased. 1 mm² can be imaged in roughly 1 minute and a square centimeter cab be imaged in 1 hour 40 minutes. The barcode for the 167 gene experiment described above is encoded in a 16-bit code, and 16 cycles of detection were performed. The chemistry is roughly performed in 1 hours and the total 16 detection cycles including chemistry are run in roughly 45 hours for 1 cm².

For comparison, a similar method osmFISH takes about 8 hours to perform the chemistry and 14 hours per imaging cycle. The area that could be imaged is considerably lower, and these 14 hours were needed to image just 3.8 mm², so that the full 13 round experiment for 33 genes took 2 weeks to complete. Examining RNA from 167 genes would take several months.

### Example 6

### Primary Oligonucleotide Probes

The primary oligonucleotide probes used in these experiments were produced using the method described in Moffitt et al (Methods Enzymol. 2016 572: 1-49). In brief, the primary oligonucleotide probes were made from a pool of oligonucleotides, where each oligonucleotide is a low concentration. Such pools can be ordered from Twist Bioscience or Agilent Technologies. These oligonucleotides are amplified by PCR after which the PCR product is in vitro transcribed into RNA of the opposite strand. To make single-stranded DNA probes, the RNA is reverse transcribed into the cDNA, and the RNA is degraded to make a pool of single-stranded DNA primary oligonucleotide probes.

An amine modified primer can be used for the last step, i.e., the reverse transcription step, so that the primary oligonucleotide probes produced by probes have an amine modification (which would be at or near the 5′ end of the oligonucleotides, e.g., within 20 bases of the 5′ end). In typical design, the primary oligonucleotide probes have a 5′ tail that hybridizes with one or more labeled probes, a central region that hybridizes with an RNA, and a 3′ tail that hybridizes with one or more labeled probes. In this example, one of the tails has the amine modification. If amine modified primary oligonucleotide probes are used, they can be attached to the substrate after hybridization with the RNA on the substrate, for example, using paraformaldehyde (PFA). This increases the stability of probes to remain in place and enhances the efficiency by which the barcodes is decoded. In this example, the amine group used is a 5′ amine with a carbon-6 spacer referred to as “5AmMC6”. The modification has minimal effect on the yield of the reverse transcription. The positioning of the amine group in the primer can vary, and length and type of spacer are not critical. For example, this method could also work with the amine modification towards the 3′ of the primer, without the carbon spacer or with another spacer and with amine modified bases that are internal in the sequence. Furthermore, this improvement could also potentially work with other modifications, e.g., acrydite, biotin, carboxy, azide, etc., as desired.

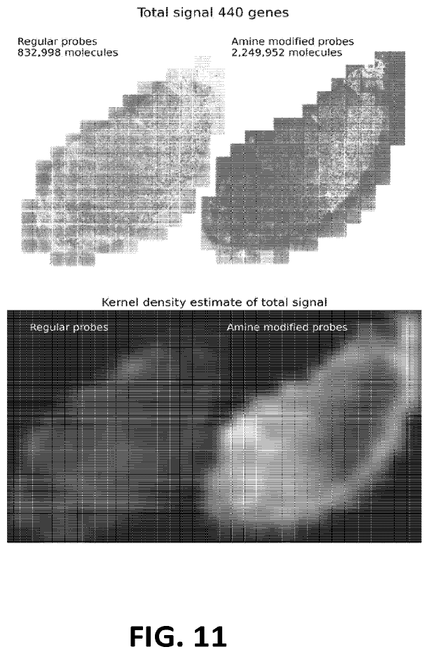

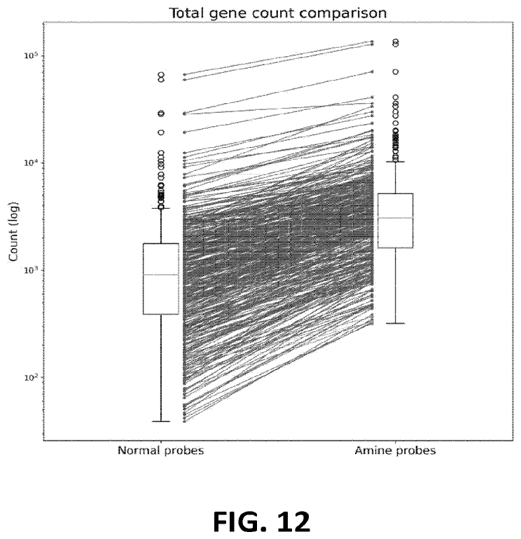

FIGS. 11 and 12 illustrate comparisons of data obtained using primary probes that have and do not have an amine modification, where data obtained using non-amine modified probes are shown on the left, and data obtained using amine modified probes are shown on the right. The two experiments are run on consecutive tissue sections of one human glioblastoma brain tumor sample. More molecules can be detected using the amine modified probes. FIG. 11 top panel shows a scatter plot of the signal of 440 genes, where every dot is one detected molecule (Left without the amine modification and right with the amine modification). 2.7 times more molecules were detected using amine-modified probes, as shown by an increase int the counts (which increased from 832,998 to 2,249952 molecules total). The bottom panel of FIG. 11 shows a kernel density estimate for the total detected molecules of both samples. This plot represents the density of molecules between the non-amine modified probes on the left and the amine modified probes on the right. FIG. 12 compares the gene count, where the numbers of probes detected in a sample treated with amine probes were greater than the corresponding numbers of probes detected in a sample treated with normal (e.g., non-amine) probes.

### Example 7

### Probabilistic Barcode Caller

A probabilistic barcode caller to calculate the posterior probability that a spot i has barcode bⱼ, where the barcode is defined by bⱼ=1 if the barcode is positive (e.g., present) in cycle j, and bⱼ=0 if the barcode is negative (e.g., absent). For this, the per-cycle probability P(Bᵢⱼ=bⱼ) is calculated first, and then the product is taken over all the cycles. For preparing the probabilistic barcode caller, a set of reference spots with known barcodes is provided. The spots might be obtained in each experiment by using a simple thresholding on the intensity values and selecting the spots with exact barcode matches. For this, no error-correction should be applied.

For the hybridization cycles of an experiment, let Xᵢⱼ be a stochastic variable representing the signal intensity of spot i in cycle j, and let x be an observed intensity. The intensity values can be raw values from the images, or can be normalized and/or background-corrected according to best practices in image preprocessing. With a large number of reference spots, the probability distribution of the intensities P(Xᵢⱼ=x) is obtained. In addition, for each cycle, the reference spots are partitioned into two groups by separating those reference spots that have barcodes that are known to be negative (e.g., absent) in the cycle, and those that have barcodes that are known to be positive (e.g., present). Then for each cycle, the probability distributions of the intensities conditional on the barcode being positive or negative in the cycle are calculated:

P(Xᵢⱼ=xⱼ|Bᵢⱼ=b), where b∈{0,1}

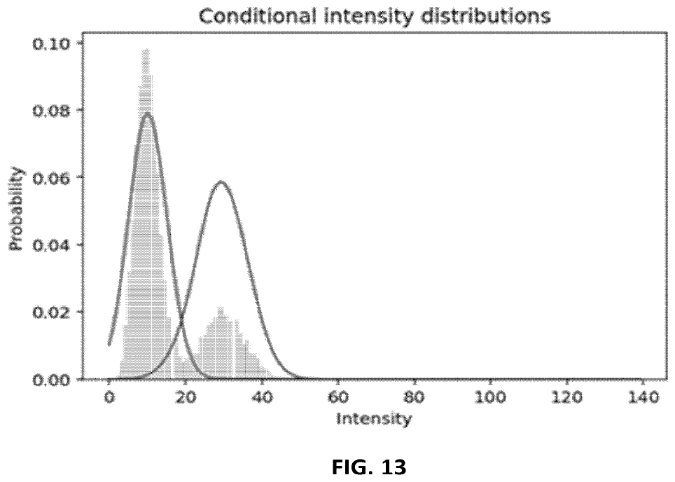

Example probability distributions obtained using simulated data are shown in FIG. 13.

Then, for a given observed intensity xᵢⱼ for a spot (which may be from an independent sample or the reference set), the posterior probability that the barcode of that spot was positive or negative in that cycle is calculated. In some embodiments, the posterior probability is calculated using the B ayes theorem. For example, an equation defining the posterior probability P(Bᵢⱼ=b) is as follows:

$$P ( {B}_{i j} = b ) = \frac{P ( {X}_{ij} = {x}_{i j} \vert {B}_{i j} = b ) P 0 ( {B}_{i j} = b )}{P ( {X}_{ij} = {x}_{i j} )}$$

where P0 is an a priori probability that the barcode is positive or negative in the cycle, which can be set to the fraction positives and negatives across all valid barcodes.

The probability of a given barcode is calculated by multiplying the posterior probabilities across the cycles:

$${\prod }_{j} P ( {B}_{i j} = {b}_{j} )$$

This calculates the probability of every possible barcode, not just those that are valid. In some embodiments, a priori probabilities for invalid barcodes are set to zero to prevent mapping to an invalid barcode. In some embodiments, the probabilities are normalized for the valid barcodes so that the total probability is 1.

In some cases, the intensity distribution in each cycle is independent of the barcode identity. For example, all different barcodes that are positive (or negative) in a cycle have the same intensity distribution. In some embodiments, the joint intensity distribution across all cycles is used.

### Example Implementations

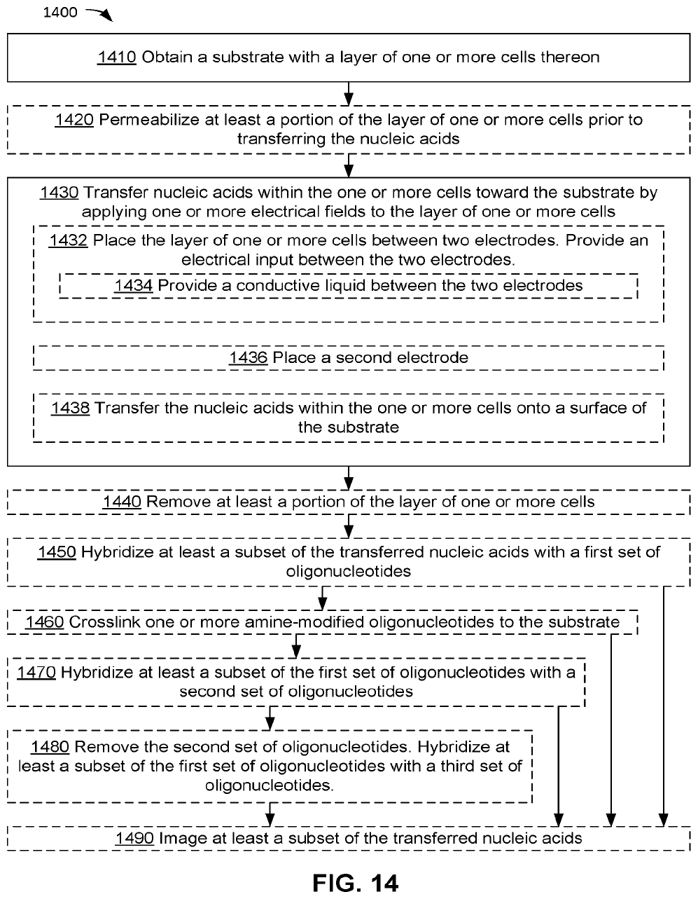

FIG. 14 is a flow diagram illustrating a method 1400 of transferring nucleic acids in accordance with some embodiments.

The method 1400 includes (1410) obtaining a substrate with a layer of one or more cells thereon (e.g., FIG. 1A). For example, the method 1400 may include receiving the substrate with the layer of one or more cells thereon. In some embodiments, obtaining the substrate with the layer of one or more cells includes placing the layer of one or more cells on the substrate (e.g., FIG. 1A). In some embodiments, the layer of one or more cells is a section of a biological tissue (e.g., a layer of tissue obtained by cutting the tissue with a microtome).

In some embodiments, the nucleic acids include one or more selected from a group consisting of ribonucleic acid (RNA) or deoxyribonucleic acid (DNA). For example, the nucleic acids may include RNA, DNA, or both. In some cases, the nucleic acids include RNA without DNA (e.g., RNA only). In some other cases, the nucleic acids include DNA without RNA (e.g., DNA only).

In some embodiments, the deoxyribonucleic acid includes one or more selected from a group consisting of genomic DNA (gDNA) or complementary DNA (cDNA). For example, the DNA may include gDNA, cDNA, or both. In some cases, the DNA includes gDNA without cDNA (e.g., the DNA includes gDNA only). In some other cases, the DNA includes cDNA without gDNA (e.g., the DNA includes cDNA only).

In some embodiments, the method 1400 includes (1420) permeabilizing at least a portion of the layer of one or more cells prior to transferring the nucleic acids. For example, one or more permeabilization agents (e.g., detergent, solvent, etc.) are provided to the one or more cells to induce permeabilization.

The method 1400 also includes (1430) transferring nucleic acids within the one or more cells toward the substrate by applying one or more electrical fields to the layer of one or more cells (e.g., steps 104, 106, and 108, FIG. 1B). In some embodiments, the method 1400 includes transferring nucleic acids within the one or more cells toward a surface of the substrate facing the layer of one or more cells (e.g., the top surface of the substrate 4 shown in FIG. 1B) by applying one or more electrical fields to the layer of one or more cells.

In some embodiments, the method 1400 includes (1432) placing the layer of one or more cells between two electrodes (e.g., steps 100 and 102, FIG. 1B). The method 1400 also includes providing an electrical input between the two electrodes (e.g., step 104, FIG. 1B). For example, at step 104 of FIG. 1B, a higher voltage is provided to the electrode 12 and a lower voltage is provided to the electrode 14 (e.g., a positive voltage is provided to the electrode 12 and a negative voltage is provided to the electrode 14, a positive voltage is provided to the electrode 12 and the ground voltage is provided to the electrode 14, or a negative voltage is provided to the electrode 14 and the ground voltage is provided to the electrode 12).

In some embodiments, the substrate is coupled with a first electrode (e.g., electrode 12). In some embodiments, the first electrode is integrated with the substrate (e.g., the substrate comes with a layer of the first electrode). In some embodiments, the method 1400 includes placing the first electrode on the substrate (e.g., mechanically coupling the first electrode with the substrate).

In some embodiments, the method 1400 includes (1434) providing a conductive liquid (e.g., conductive liquid 16, FIG. 1B) between the two electrodes.

In some embodiments, the method 1400 includes (1436) placing a second electrode (e.g., electrode 14). In some embodiments, the second electrode is placed on a side of the layer of one or more cells opposite to the substrate (e.g., in FIG. 1B, the electrode 14 is placed on the opposite side of the sample 2 from the substrate 4).

In some embodiments, transferring the nucleic acids within the one or more cells toward the surface of the substrate facing the layer of one or more cells includes (1438) transferring the nucleic acids within the one or more cells onto the surface of the substrate facing the layer of one or more cells. For example, as shown in step 108 (FIG. 1B), the nucleic acids from the one or more cells are transferred onto the surface of the substrate 4.

In some embodiments, the method 1400 includes, after transferring the nucleic acids, (1440) removing at least a portion of the layer of one or more cells.

In some embodiments, the method 1400 includes (1450) hybridizing at least a subset of the transferred nucleic acids with a first set of oligonucleotides. In some embodiments, the first set of oligonucleotides includes labeled probes (e.g., oligonucleotides coupled with one or more labels). For example, in some embodiments, a respective oligonucleotide of the first set of oligonucleotides is coupled to a corresponding first label. In some embodiments, a combination of the respective oligonucleotide and the corresponding first label does not occur naturally (e.g., the respective oligonucleotide and the corresponding first label do not naturally occur together).

In some embodiments, the first set of oligonucleotides includes primary oligonucleotides (e.g., oligonucleotides that are not covalently linked to labels, oligonucleotides without labels, etc.).

In some embodiments, the first set of oligonucleotides includes one or more amine-modified oligonucleotides (e.g., an oligonucleotide with 5′ amine). In some embodiments, the method 1400 includes (1460) crosslinking the one or more amine-modified oligonucleotides, hybridized to at least a subset of the transferred nucleic acids, to the substrate.

In some embodiments, the method 1400 includes (1470) hybridizing at least a subset of the first set of oligonucleotides with a second set of oligonucleotides (e.g., the labeled probes).

In some embodiments, a respective oligonucleotide of the second set of oligonucleotides is coupled to a corresponding second label. In some embodiments, a combination of the respective oligonucleotide and the corresponding second label does not occur naturally (e.g., the respective oligonucleotide and the corresponding second label do not naturally occur together).

In some embodiments, the method 1400 includes (1480) removing the second set of oligonucleotides and hybridizing at least a subset of the first set of oligonucleotides with a third set of oligonucleotides that is distinct from the second set of oligonucleotides (e.g., FIG. 1A).

In some embodiments, the method 1400 includes (1490) imaging at least a subset of the transferred nucleic acids. For example, an image of the substrate or the transferred nucleic acids is obtained by an imaging device (e.g., a camera, which may be integrated with a fluorescence microscope). In some cases, the image may show a distribution of the first set of oligonucleotides hybridized to the transferred nucleic acids (e.g., in case where the first set of oligonucleotides are labeled and bound to the transferred nucleic acids). In some cases, the image may show a distribution of the second set of oligonucleotides hybridized to the first set of oligonucleotides (e.g., in case where the second set of oligonucleotides are labeled and bound to the first set of oligonucleotides).

In some embodiments, the substrate is planar (e.g., the substrate may be a microscope slide). In some embodiments, the substrate is not planar (e.g., the substrate has a curved surface on which the layer of one or more cells may be placed).

In some embodiments, the substrate is optically transparent (e.g., the substrate is made of glass, transparent plastic, etc.). In some implementations, the substrate is optically transparent to a visible light (e.g., for fluorescence imaging). In some implementations, the substrate is optically transparent to an ultraviolet light (e.g., UV illumination). In some implementations, the substrate is optically transparent to an infrared light (e.g., for probes with infrared dyes). In some implementations, the substrate is optically transparent to two or more of: a visible light, an ultraviolet light, or an infrared light.

In some embodiments, the method 1400 is performed using an apparatus that includes a mount (e.g., the mount 18, FIG. 1B) for receiving a substrate and an electrical source (e.g., the electrical source 20, FIG. 1B) positioned adjacently to the mount for providing one or more electrical fields in a direction that is substantially perpendicular to the substrate (e.g., the electrical field shown in FIG. 1B is substantially perpendicular to the substrate 4). In some cases, the electrical field is perpendicular to the substrate. In some cases, the direction of the electrical field forms an angle that is greater than 20 degrees, 25 degrees, 30 degrees, 35 degrees, 40 degrees, 45 degrees, 50 degrees, 55 degrees, 60 degrees, 65 degrees, 70 degrees, degrees, 80 degrees, or 85 degrees, relative to the substrate. In some embodiments, the electrical field that is not parallel to the substrate is used, because an electrical field parallel to the substrate is not effective at transferring the nucleic acids toward the substrate.

In some embodiments, the substrate is coupled with two electrodes (e.g., the substrate 4 of FIG. 1B directly coupled with the electrode 12 and indirectly coupled with the electrode 14). The electrical source is electrically coupled with the two electrodes for providing an electrical input (e.g., provides a voltage or a current so that the one or more electrical fields are provided).

Some embodiments may be described with respect to the following clauses:
Clause 1. A method, comprising:
receiving a substrate with a layer of one or more cells thereon; and
transferring nucleic acids within the one or more cells toward a surface of the substrate facing the layer of one or more cells by applying one or more electrical fields to the layer of one or more cells.
Clause 2. The method of clause 1, wherein:
the nucleic acids include one or more selected from a group consisting of ribonucleic acid (RNA) or deoxyribonucleic acid (DNA).
Clause 3. The method of clause 2, wherein:
the deoxyribonucleic acid includes one or more selected from a group consisting of genomic DNA (gDNA) or complementary DNA (cDNA).
Clause 4. The method of any of clauses 1-3, including:
placing the layer of one or more cells between two electrodes; and providing an electrical input between the two electrodes.
Clause 5. The method of clause 4, further comprising:
providing a conductive liquid between the two electrodes.
Clause 6. The method of any of clauses 1-5, wherein:
the substrate is coupled with a first electrode.
Clause 7. The method of clause 6, further comprising:
placing a second electrode on a side of the layer of one or more cells opposite to the substrate.
Clause 8. The method of any of clauses 1-7, wherein:
transferring the nucleic acids within the one or more cells toward the surface of the substrate facing the layer of one or more cells includes transferring the nucleic acids within the one or more cells onto the surface of the substrate facing the layer of one or more cells.
Clause 9. The method of any of clauses 1-8, further comprising:
after transferring the nucleic acids, removing at least a portion of the layer of one or more cells.
Clause 10. The method of any of clauses 1-9, further comprising:
hybridizing at least a subset of the transferred nucleic acids with a first set of oligonucleotides.
Clause 11. The method of clause 10, wherein:
a respective oligonucleotide of the first set of oligonucleotides is coupled to a corresponding first label, wherein a combination of the respective oligonucleotide and the corresponding first label does not occur naturally.
Clause 12. The method of clause 10 or 11, further comprising:
hybridizing at least a subset of the first set of oligonucleotides with a second set of oligonucleotides.
Clause 13. The method of clause 12, wherein:
a respective oligonucleotide of the second set of oligonucleotides is coupled to a corresponding second label, wherein a combination of the respective oligonucleotide and the corresponding second label does not occur naturally.
Clause 14. The method of clause 12 or 13, wherein:
the first set of oligonucleotides includes one or more amine-modified oligonucleotides.
Clause 15. The method of clause 14, further comprising:
crosslinking the one or more amine-modified oligonucleotides, hybridized to at least a subset of the transferred nucleic acids, to the substrate.
Clause 16. The method of any of clauses 12-15, further comprising:
removing the second set of oligonucleotides; and
hybridizing at least a subset of the first set of oligonucleotides with a third set of oligonucleotides that is distinct from the second set of oligonucleotides.
Clause 17. The method of any of clauses 1-16, further comprising:
imaging at least a subset of the transferred nucleic acids.
Clause 18. The method of any of clauses 1-17, further comprising:
permeabilizing at least a portion of the layer of one or more cells prior to transferring the nucleic acids.
Clause 19. The method of any of clauses 1-18, wherein:
the substrate is planar.
Clause 20. The method of any of clauses 1-19, wherein:
the substrate is optically transparent.
Clause 21. A method for imaging RNA transferred from a sample to a substrate, comprising:
placing a sample comprising cells and having at least one planar surface on a planar substrate;
transferring RNA from the sample onto the planar substrate to produce an RNA blot in which the RNA is immobilized on the substrate;
removing the sample from the substrate;
hybridizing the RNA blot with a set of oligonucleotides that hybridize to different sites in the same RNA species; and
reading the blot to obtain an image showing the binding pattern of the hybridized oligonucleotides.
Clause 22. The method of clause 21, wherein the RNA is transferred to the planar substrate by electrophoresis.
Clause 23. The method of clause 21 or 22, wherein the RNA is transferred from the sample to the planar sample by done by:
   i. placing the sample on a planar, optically transparent, conductive substrate,
   ii. positioning a planar electrode opposite to the sample, and
   iii. applying a voltage across the substrate and electrode when the sample immersed in a conductive liquid, thereby moving the RNA in the sample to the substrate.
Clause 24. The method of clause 23, wherein the conductive liquid optionally comprises a denaturant (e.g., urea).
Clause 25. The method of clause 23, wherein the voltage is in the range of 25 V/cm to 300 V/cm.
Clause 26. The method of any prior clause, wherein the substrate is a transparent (e.g., glass) slide coated in a transparent conductive metal oxide (TCO), or a thin layer of gold, titanium with gold, chromium with gold.
Clause 27. The method of clause 26, wherein the TCO coating is an indium tin-oxide (ITO), aluminum-doped zinc oxide (AZO), indium-doped cadmium oxide ICO), molybdenum indium oxide, (MIO), gallium zinc oxide (GZO), fluorine-doped indium oxide (IFO) or indium zinc oxide (IZO) coating.
Clause 28. The method of any prior clause, wherein the substrate is a glass slide that has a transparent conductive metal oxide (TCO) coating, wherein the sample is placed directly or indirectly onto the TCO coating or a TCO coating that has been modified.
Clause 29. The method of any prior clause, wherein the
   hybridizing the reading and reading steps are done by:
   hybridizing the RNA blot with a set of unlabeled primary oligonucleotides that hybridize to different sites in the same RNA species and have a tail, hybridizing the blot with labeled probe that hybridizes to the tail of the unlabeled primary oligonucleotides; and
reading the blot to obtain an image showing the binding pattern of the hybridized labeled probes.
Clause 30. The method of clause 29, wherein the unlabeled primary oligonucleotides are amine-modified, and the method comprises crosslinking the amine-modified primary oligonucleotides to the substrate after they have been hybridized to the RNA blot but before hybridization of the labeled probes.
Clause 31. The method of any prior clause, wherein the method comprises:
(a) obtaining multiple sets of unlabeled primary oligonucleotides and multiple labeled probes, wherein:
i. each primary oligonucleotide comprises a sequence that hybridizes to a particular RNA species and a tail sequence that does not hybridize to the RNA;
ii. the different sets of primary oligonucleotides hybridize to different RNA species;
iii. the labeled probes hybridize to the tails of the primary oligonucleotides;
iv. at least some of the labeled probes hybridize to multiple sets of primary oligonucleotides; and
v. each set of primary oligonucleotides hybridizes with a unique combination of labeled probes;
(b) placing a sample comprising cells and having at least one planar surface on a planar substrate;
(c) transferring RNA from the sample onto the planar substrate to produce an RNA blot, wherein in the blot the RNA is immobilized on the substrate;
(d) hybridizing the RNA blot, en masse, with the unlabeled primary oligonucleotides,
(e) hybridizing the blot with a subset of the labeled probes;
(f) reading the blot to obtain an image showing the binding pattern of the labeled probes hybridized in (e);
(g) inactivating or removing the subset of labeled probes hybridized in (e), without removing the primary oligonucleotides;
(h) repeating steps (e)-(f) using a different subset of the labeled probes, each repeat followed by step (g) except for the final repeat, to produce a plurality of images of the sample, each image corresponding to a subset of labeled probes hybridized in (e);
(i) analyzing the same site in the plurality of images to identify which labeled oligonucleotides hybridized to the site; and
(j) identifying the species of RNA at the site using the labeled oligonucleotides identified in (i).
Clause 32. The method of any prior clause, wherein the surface of the planar substrate upon which the sample is placed comprises a polycationic adhesive.
Clause 33. The method of clause 32, wherein the polycationic adhesive is polylysine.
Clause 34. The method of any prior clause, wherein the surface of the planar substrate upon which the sample is placed comprises oligo(d)T.
Clause 35. The method of any prior clause, wherein the method comprises chemically cross-linking the RNA to the substrate.
Clause 36. The method of any prior clause, wherein the substrate comprises one or more fiducial markings for image registration.
Clause 37. The method of clause 36, wherein the fiducial markings are fluorescent beads that are deposited on the substrate.
Clause 38. The method of any of clauses 29-37, wherein the different RNA species are encoded by different genes.
Clause 39. The method of any of clauses 29-38, wherein each set of primary oligonucleotides comprises at least 1, at least 25, or at least 30 (e.g., 30-300) primary oligonucleotides.
Clause 40. The method of any of clauses 29-39, wherein step (a) comprises obtaining at least 30 sets (e.g., 30-3000 sets) of unlabeled primary oligonucleotides.
Clause 41. The method of any of clauses 29-40, wherein step (h) comprises repeating steps (e)-(f) at least 4 times (e.g., 25-50 times).
Clause 42. The method of any of clauses 29-41, wherein a subset of labeled probes of (e) is one labeled probe.
Clause 43. The method of any of clauses 29-42, wherein a subset of labeled probes of (e) is multiple labeled probe that are distinguishably labeled.
Clause 44. The method of any of clauses 29-43, wherein the labeled probes each comprise: (a) an oligonucleotide and (b) a label, wherein (a) and (b) are connected by to each other via a cleavable linker.
Clause 45. The method of clause 44, wherein the cleavable linker comprises a disulfide bond.
Clause 46. The method of any of clauses 29-45, wherein in step (g) the probes are inactivated by addition of a reducing agent (e.g., (3-mercaptoethanol, TCEP) that releases the label from the oligonucleotide.
Clause 47. The method of any prior clause, wherein the sample is an unfixed tissue section or cell monolayer.
Clause 48. The method of any prior clause, wherein the sample fresh tissue, fresh frozen tissue, or a fixed tissue.
Clause 49. The method of any prior clause, wherein the cells in the sample are permeabilized between steps (b) and (c) optionally in the presence of a reducing agent (e.g., DTT).
Clause 50. The method of any prior clause, wherein the method further comprises staining and imaging the planar sample.
Clause 51. The method of any of clauses 29-50, wherein the method further comprises registering the images produced in step (h).
Clause 52. The method of any of clauses 29-51, wherein step (j) comprises use of a look-up table.
Clause 53. An apparatus, comprising:
a mount for receiving a substrate; and
an electrical source positioned adjacently to the mount for providing one or more electrical fields in a direction that is substantially perpendicular to the substrate.
Clause 54. The apparatus of clause 53, wherein:
the substrate is coupled with two electrodes; and
the electrical source is electrically coupled with the two electrodes for providing an electrical input.
Clause 55. An apparatus configured for performing the method of any of clauses 1-52.

In some embodiments, the embodiments described with respect to clauses 21-52 include one or more features described with respect to clauses 1-20. In some embodiments, the embodiments described with respect to clauses 1-20 include one or more features described with respect to clauses 21-52. For brevity, such details are not repeated herein.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. A method, comprising:
receiving a substrate with a layer of one or more cells thereon; and
transferring nucleic acids within the one or more cells toward a surface of the substrate facing the layer of one or more cells by applying one or more electrical fields to the layer of one or more cells.

2. The method of claim 1, wherein:
the nucleic acids include one or more selected from a group consisting of ribonucleic acid (RNA) or deoxyribonucleic acid (DNA).

3. The method of claim 2, wherein:
the deoxyribonucleic acid includes one or more selected from a group consisting of genomic DNA (gDNA) or complementary DNA (cDNA).

4. The method of claim 1, including:
placing the layer of one or more cells between two electrodes; and
providing an electrical input between the two electrodes.

5. The method of claim 4, further comprising:
providing a conductive liquid between the two electrodes.

6. The method of claim 1, wherein:
the substrate is coupled with a first electrode.

7. The method of claim 6, further comprising:
placing a second electrode on a side of the layer of one or more cells opposite to the substrate.

8. The method of claim 1, wherein:
transferring the nucleic acids within the one or more cells toward the surface of the substrate facing the layer of one or more cells includes transferring the nucleic acids within the one or more cells onto the surface of the substrate facing the layer of one or more cells.

9. The method of claim 1, further comprising:
after transferring the nucleic acids, removing at least a portion of the layer of one or more cells.

10. The method of claim 1, further comprising:
hybridizing at least a subset of the transferred nucleic acids with a first set of oligonucleotides.

11. The method of claim 10, wherein:
a respective oligonucleotide of the first set of oligonucleotides is coupled to a corresponding first label.

12. The method of claim 10, further comprising:
hybridizing at least a subset of the first set of oligonucleotides with a second set of oligonucleotides.

13. The method of claim 12, wherein:
a respective oligonucleotide of the second set of oligonucleotides is coupled to a corresponding second label.

14. The method of claim 12, wherein:
the first set of oligonucleotides includes one or more amine-modified oligonucleotides.

15. The method of claim 14, further comprising:
crosslinking the one or more amine-modified oligonucleotides, hybridized to at least a subset of the transferred nucleic acids, to the substrate.

16. The method of claim 12, further comprising:
removing the second set of oligonucleotides; and
hybridizing at least a subset of the first set of oligonucleotides with a third set of oligonucleotides that is distinct from the second set of oligonucleotides.

17. The method of claim 1, further comprising:
imaging at least a subset of the transferred nucleic acids.

18. The method of claim 1, further comprising:
permeabilizing at least a portion of the layer of one or more cells prior to transferring the nucleic acids.

19. The method of claim 1, wherein:
the substrate is planar.

20. The method of claim 1, wherein:
the substrate is optically transparent.
